SEQUENCE LISTING (18 sequences)

SEQ ID NO: 1 - DNA, 16419 bp, Felis catus
ctgagcccattggcttctggtcacctgataatgctcttactgcagacaagacctctgtcccgcggtgcacttgttgcccagaactctccgttgctgccctggggcctcctcgcggcctcggattccagctcagcctctcccgtcgcctggctcatggcgctactcttctccctaatcctcggtgagtatctcgggccctgctggctgaggcttcagatctccccagaaccccctgcccagctggtcccactccctctcaaggagtgacttctgctctcctgccccttctgtgtccaacagtgttctaaacccaaagccactcagtccacacccaatttagaatccactgtgatccccagaggttttattttttttcctattactttctatttgccctgacaaatttataatagtctatcatttcccctgttgcataaatttccccaaatgtactcttttttttttgaaaaagacaagtgaaccatgcgtagcagcaggtgggggaagaaaaggaagtagaaccacaggcaaacaccttccacagaccacgctgtccagtttcctccaaaaagtgtttcatgcgggatttcccagtttcttatcatccagattcattcacagtaaatgggattttggagtcaaagggacccgggtttcaattttcgtcctttgatttcttggctgtctgaattgttcagttaccctctctctctggaatcttgttttcatctatggaatgaggagaatattatgttctctagaggcctgtgggagacttttcacgaattaagaagcttggggcatccaacaatgcctacgaggtcccttccctaactaatcacacccatttttaagttttatttattcatttggagagagagagggagagatggagagagagagggagagagagaggggatcccaagcagcctctactcagatgcagggctcagactcatgagccgcgagatcatgacctaagctgaaatcatgagttggatgcccaactgcctgggccaccaggcatcccaatcttacccactttgtattgtcttgatgttctggtgcatcactggagcttgcactgggctctcaaccaggcattgatgtgctctgtacaggatgctggccactctcctaaactccattgtgtcccctgtatcacccaagagcgggccggcccatcacatgcatcctaccctgtcttcccgactttccttcggccccacctccttggtgaagcctctccggtccggtccccagcagtcgtctactctgccccagagttttcaagtcactcttgggctctgtcactttcacacctgcatgtcagggtcgacacgctcagtttcctctccgtgtgcacatgcttcgtgctctttactggagatgtggggggttacgtgtgatacgatctgcagttttagacagacagctgcggacaagcggccacatcctgaaggccaagggcttagggttgttaagaaactacttggcaaatagtccagaacaagccaatgaaattcttggatctgcccgaaaggctctagaccaaagatattagctccttcacaaacattccacaagatgtggggagggaaaaactgttctgcactgcagaatgcctagggccttggggaccactcggggaccacaggtggacacagggttcaggactcttggcagcattagaagggagcccaatgcgtggaggatcccggccacccagaggtcctgcgggaagaaactatgatgtcctaagacgtgccggtgcttaaggctggggattgggaagcaaatacagagctgctttctgacctgttgagcccccgggggccggcaaacagcctccaagctaagggtggtgtttacatttttcaacggtgggaaaaaatatccaaagaagaatgatattttgtgcaccgaagtctgaatctcagagtctatgaacaaaggttggttgcgacatggccatgctctttgtgctcgttcggacatggcaactctcacgctacagtgacaaggttgagtagtggtgacagagaccgtgtggctacaaagcccaggttatttgcgatctggccctttacagaaacagtacgctggctgtggtcccaatgcagcgccagtgaacgtagacgatccctcccatttcccccgctctgagctgccgtggtggtgtcgctgccttctgcaccagagcccgttccggggacctctgacacctcttcctgtggagcccagaggctgcacaacacccctggggcagagccaacgttgcgtgttcggggaacatgatgggctgtggcagaccggtctttgtggttcccagccactgtccgtgtgtccttgcaggggaaggtccccacaggaccgagtctggaggctctgtacccacactttagctgccccacaaagtctagcaaaaaatttctagacagtcgtgttgtttgaacaaagccttggaaatggactgtatgacctcactcaacttccttcttctctccccagccatttacactggacctggcattttaggtaagactttttgaggtctttgagaggcgggggggtggggggctccaagctccagcgatggaaaatttgaagcttcctggggcacggagagaggctctattcgatacagccctggtagatttccttcccggtttctcagtccgcccactcagtcttatgtggatggcttcccacctccagaacctgggagggattcgggtggcatttcccgaaaggggattccctcccacttggcctctgtggcccaagactcttgggcacctccaagcagcaggtggccttgtttggggctcggaaagcacctcacctcctcgcgggcatatctgggtcaggagcttcctcttgccctgctgggctctaccactggcgggaccctgtgtcagcctgcatggctcctttgaactagaaatgcgtcaacttgtatgcggcctgtggctggaccggcagcaggcgggctggatttcagcctccaccggcccctcgagtgagggtccttgggcaacatacgaggggaatgggtctctcaagaggaagactgatgctccagactctaggcggttggtcgtggactatcagctccttagatattaccccttgcgtgttctttgtcttgacggtagaggcttcgcgagagagggacagccactagcctctagcctctttactagagagggaccccgcatctgcagggtgcagtttgattccttctggagggctgcctggggttctcagcctgacgctcagattacacgggccttttcctgtggcttccagggctgttcttgctgctggcacagggaccactgctgtccagctttccatggcccgggacgctccccctccaccccagggttctgctctggagccatcagccaacacagtaggtcgaggacactgccggggacgcagctgcctcgatggtcccccagcccttctccctggctttcagatctcttcgcagttgggtttcttggacactaacactgtttctttcttacatatttcttctttcctgggaggtgttctacgcctcctttctgtggaaaggaaaactcacttagagccagaattttacaattcagaatcagataatgtgtgcagccagcatcccggaggacacaataatgcctgtctaaacgctaatcttgcaatgaaatgctgtttctgggcgactcctctagggttatctttatttgtaggctgcaaacttaggtgtgcaaaaatgaaaaccttgcttcgttgttttgtagttacacttaaatctctgtctctagcctccatgcctcacattgtttccctcaaaagacacatggaggaaaaggagagacacaaaagcaaatgagcaaaccaaaggtcaacttccaaagcactttgaagtcagccagctcagagctttcccactcagccatgtcttctggggttgaatgcaggttctagaggatttgtgtgccacagaggcagtgggagatctgctccttcgaggtcatattccattcctgcaggtgcctgggtcacccacgtgtcccacgtggtcacaggcgacaatgctcgcatcctggttggtcacagaccagcaggcacgtgcggtcccgccaggtgccccagacctctctaggtcaaccgccccatcccgtggaagcaaggctgactcagcttttctgtgccacgtcgtcactcggaggctgcaggagacccgctggggttgcctcagatgtcttaactataatgtccttgaggacagcagtgtggacagtcttgttggtcactgttctttgagttcatgaacatagtatgtgctcaataaaagtgtgttgcctgatgagctcgtctctgtcctccccacagccatcactctatgtctatgtcttactggaagagggagaggatttcagttattcttttccaccctcagagttgtagctgggaggcagaaacaggttctggattcctcagaaattatctggaggctctggggaggctgcctcctaccccggctgctggaaccctctggactaatgaccgaggctcagatgtgcaaaggcttggccctttgcctacttctctctgcattcttctgagctgtgttcttggacatgggaagatgtcatagggagcagcaaggtaggggagatcgttgatattgggagagagagagagtcaggttgttcagcccaaattatatctaaacatcctgcctcatctggtggccatggggaaggacagggtcaagaagaaagaatgcaacagggaacacctctacgtttgtgttctgacctggggcttccaccgtctggccacgtgagcatgtgtttgtaggtgtcttgctgtggagtttcttggcagggtaaattaaaagggagcagaatcagagcttagggatgaggtcctgaagctcctctcccccccgccttcccttttgagccctcctttcccatcccccagctttccaaacaccctctgtcagtgatggaagtttcctgagatgcctgcatcctctccgggctcctccggatgcccagccaggctcccgttcctttgcagggtctttttccagagtgcggctagtgggaggcgaccaccgctgtgaaggtcgtgtggagttgcagcaggatgacgagtgggtcaccgtgtgtgatgactactggaacatggactctgtggccgtgctgtgccgggagctgggctgtggggcggccaggaagaccatgagtggcaccgtgtatggaccagtgacaccaaaggaccaaaaagtcttcatccacctgttcagatgcaatgggatcgaagaaagcctgtctcagtgtgagagggaagatgcaatcggatgctcccatgatgaggatgcgggagccgtgtgcgagggtgagtcccacagcccctcaggccagttcacctgtctgagcccactgtggacgtgacacgtggctaactggctcctcacccttcgtgtctcacctcttcctgatcgtgggccctgcaggtgaacactaccctcacttagtattgcccgtgctctttggttgaggtaccgcattcactttagtttcagcccacgtgaagggctgcttctcccctttgtgcacgtgcacgcgcacacacacacacacacacacacacacacacgagcacgcacagatccatccaccactaaatgttggatcaagagtataacctgtgtctggagcttggcgggaggtatggggaagaaaagagccggtgaagtacgatgtatggttcccgggttttggtgcttaaaaaaaaaaagtacaggggcgcctgggtggctcaattgattaagtgtccgacttcagctcaggtcatgatctcgcggtttgtgggttctagccccgcatcgatgatctggagttttagacagctgcggacaagcggccacatcctgaaggccaagggcttagggttgttaagaaactacttggcaaatagtccagaacaagccaatgaaattcttggatctgcccgaaaggctctagaccaaagatattagctccttcaccaacattccacaagatgtggggagggaaaaactgttctgcactgcagaatgcctagggccttggggaccactcggggaccacaggtggacacagggttcaggactcttggcagcattagaagggagcccaatgcgtggaggatcccggccacccagaggtcctgcgggaagaaaaactattttatgtcctaagacgtgccggtgcttaaggctggggattgggaagcaaatacagagctgctttcctgaccctgttgacccccgggggccggcaaacagcctccaagctaaggatggtgtttacatttttcaacggtgggaaaaaatatccaaagaagaatgatattttgtgcaccgaagtctgaatctcagagtctatgaacaaaggttggttgcgacatggcaatgctctttgtgctcgttcggacatggcaactctcacgctacagtgacaaggttgagtagtggtgacaagagaccggtgtggctacaaagcccaggtattgcgatctggccctttacagaaacagtacgctggctgtggtcccaatgcagcgccagtgaacgtagacgatccctcccatttcccccgctctgagctgccgtggtggtgtcgctgccttctgcaccagagcccgttccggggaccctctgacaactcttcctgtggagcccagaggctgcaccaacacccctggggcagtagcccacccccgtttttttgttttttgtgggggtttcggggaacatgatgggactgtggcagaccggctcttgtggttcccagccactgtccgtgtggtccttgcaggggaaggtccccacaggaccgagtctggaggctctgtacccacactttagctgccccacaaagtctagcaaaaaaattctagacagtcttgtgtgtttgaacaaagccttggaaatggactgtatgatctcactcaacttccttcttctctccccagccatttacactggacctggcagttttaggtaagactttttgaggtctttgagaggcgggggggtgggggggatggaaaatttgaaagctttcctgggcacgggagggctctattcaataacagccctggtatatttccttccgcggtattctcagtccacccactcagtcttatgtggatggcttcccacctccagaacctgggagggattcggttgtggcatttcccgaaaggggattccctcccacttggcctctgtgcccaagaactcttggcacctccaaagcagccgatggacttgtttgagagggctcggaaagcacctcacctcctcgcgggcaatatctgggtgaggagcttcctcttgccctgctgggtctacaactggcgggaccctgtgtcagcctgcatggctcctttgaactgaaatgcgtcactttatgcggcctgtggctggaccggcagcaggcgggctggattcagcctccacctggcccctcgagtgagggtccttgggggcaacaatacgaggggaatggtctctaagaaggaagactgatgctccagactctaggggttggtcgtgactatgaagctcctagatattaccccttgcgtgttctttggtcttggacggtagaggctgtcgccgagagagggacagccactagcctctagcctcgttactagagagggaccccgcatctgcagggtgcagtttgattccttctggagtgctgcctggggttcctcaggcctgacgctcagatttacacgggaccttttcctgtggcttccagggctgtcttgtgctggcaccaggaccactgctgtccagctttccatggcccggacgctccccctccaccccagggttctgctctgagccatcaagccaacacggtaggtcgagccgacactgcggacgcagctgccttgatggtcccccagcccttctccctgccttagatctcttcgcagttgggtttcttggaaactaacaactgtttctttctttacattattttcttctttcctgggaggtgttctacgcctcctttctgtggaaggaaactcacttagagccagaattttacaattcagaatcagataatgtgtgcagccagctccccggatgacacaataatgcctgtctaaacgctaatcttgcaatgaaatgctgtttctgggtgactcctctagggttatctttatttgtaggcctgcaaacttaggttgcaaaggaaaaccttttgcttcgtgttttgtagttacacttaaatctctgtctctagcctccatgccctcacattgtttccctcaaaaagggacacatgaggaaaaggagagacacaaaagcaaatgagcaaaccaaaggtcaacttccaaagcactttgaagtcagccagctcagagctttcccactcagccatgtcttctgggggttgaatgcaggttctagaggattgtgtgccacagaggcagtgggagatctgctccttcgagcgtgcatattccattcctgcaggtgcctgggtcacccacgtgtcccacgtggtcataggcgacaatgctcgcatcctggtggtcgacagccagcaggcacgactgacgggtcccgccaggttgccccaggaacctctctaggtcaaccgccccatcccgtggaagcaaggctgactcagcttttctgtgccacggcgtcactcggaggctgcaggagacccgctggggttgcctcagatgtcttataactatattgtccttgaggacagcagtgtggacagtcttgttggtcactgttctttgagttcatgaacatagtatgtgctcaataaaagtgtgttgcctgattgagctcggttctctagtcctccccacagccatcactctatgtctatgtcttactggaagagggagaggatttcgtattcttttccacccctcagagttgtagctgggagcagaaacaggttctggattcctcagaaattatctggaggctctgggagaggcttgccctcctaccccggctctggaaccctctggactaatgaccgaggctatcagatgtgcaaaggcttggccctttgcctacttctctctgcattcttctgagctgtgttcttggacatgggaagatgtcatagggagcagcaaggtaggggagatcgttgatattgggagagagagagagtcaggttgttgtacagcccaaattatatctaaacatcctgcctcatctggtggccatggggaaggacagggtcaagaaaaaaagaatgcaacagggaacactctacgtttgtgggctgacatggggcctgccaccgtcgggccacgtaagcatgtgtttgtagtgtctttgctgtggagttttcttgagcagtaattaaaagggagcagaatcagagccttagggataggtcctgaagactaccatctccccccccttccctgttgagccctcctttcccatcccccagctttccaaacaccctctgtcagtgatgggaagtttcctgagatgcctgcaatcctctccgggctcctccggatggcccagccaggctcccggtccttgggcagggtcttttttccagagtgcggctagtgggaggcgaccccgctgggaaggtcggggagttgcagcaggatgacaaggggtcccggggtgtgatactactgaactggactcggtggccgtgctggcggagcatggctgtggggcggcaggaagaccatgagggcaccggtgatggaccagtgacaccaaaggaccaaaaagtcttcatccacctgtgcaatgcaatgggatgcgagaagcctgtctcagtgcgagagggaagatgcaatcggatgctcacatgtttaaggattgcgggagccggtgtgcgagcagtgaagtaccacagcccctcgggcccgttccctgtcctgagcccactgggacgcgacacgcggtaactggtcctcacccttcgtgtctcccacctcttcctgatcgtgggccctccggtgaacactacactcacttagtagtgccgcggctctttggttgaggtaccgcatcaccttagtttcagcccccgtgaaagggcttccttcctccctttgtgcacgtcaagtgcgcgcgcgcacacacacacacacacacacacacacacacgagcacgcacagatccatccaccactaaatgttggatcaagagtataaaacctgtgtctggagcttggcgggagtatggggagaaaagcagcggtgaagtacgatgtatggttccgggttttggtgcttaaaaaaaaaaagtacaggggcgcctgggtggctcaattgattaagtgtccgacttcagctcaggtcatgatctcgcggtttgtgggttctagccccgcatcagatggatctggagttttagacaggctgcgacaacagccggccacatcctgaaggccaagggcttagggttgttaagaaactacttggcaaatagtccagaacaagccaatgaaattcttggatctgcccgaaaggctctagaccaaagatattagctccttcacaaacattccacaagatgtgggagggaaaaaactgttctgcactgcagaatgcaagttagggcctcgggcgaccactggggaccacaggtggacacagggttcaggactctggcagcattagaagggagcccaatgcgtggaggatcccggccacccagaggtcctgcgggaagaaactatgatgtcctaaagacgctgccggtgcttaaggctggggattgggaagcaaaaacgagctgctctgacctgtgaagcccccgggggccggcaaacagcctccaagctaaggatggtgtttacatttttcaacggtgggaaaaaatatccaaagaagaaatgcatattttgatatgcaaccgaagtctgaatctcagagtctatgaacaaaggttggttgcgacatatgccatgcgctttgttggcctcgttcggacatggcaactctcacgctacagtgacaagttgagtagtggttgacgagaccgtgttggccttacaaagcccaaggttatttgccgatctggccctttacagaaaacagtacgctggctgtggtcccaatgcagcgccagtgacgtagacgattccctcccatttccccgctctgagctgccgtggtggtgtcgctgccttctgcaccagagcccgttccggggacctctgacacctcttcctgtggagcccagaggctgcacaacacccctggggcagagccaacgttgtgtgttacttgcggaacatgatggggctgtgcagaccgtcctttgatggttcccaggcccactggtccgtgtgtccttgcaggggaaggtccccacaggaccgagtctggaggctctgtacccacactttagctgccccacaaagtctagcaaaaaatttctagacagtcgtgtgtttgaacaaagccttggaaatggactgtatgatccacttaacttccttcttctctccccagccattacactggacctggcattttaggtgagactttttgaggtccttgagaggcgcggggttgggtggggatcggaaaattaagcttgggcacggagagaggctctattcgatacagccatggtagatttccttcccggtttctcagtcacccacatcagtaacttttatgtggatggcttcccacctccagaacctggagggaattcgagtggtggcatattcaacgaaggggattccctccacttggcctctgtggcccaagactcttgggcacctccaatcagcagtggccttgtttgtgctcgggaaagcacctcacctcctcgcggcatatctgggtgaggagcttcctctgccctgctgggctctaccactggcgggaccctgtgtcagcctgcatggctcctttgaactagaaatgcgtcaacttgtatgcggcctgtggctgaccggcagcaggcgggctggatttcagctccaccggcccctcgagtgagggtccttgggcaactacgaggggaaatgggtctctcaagaggaagactgatgctccagactctagggggttggtcgtggactatcagctccttagatattaccccatgcgtgttctttgtcttgacagtagaggcttcgcgagagagggacagcactagcctctagcctctttacttagagaggcgaccccgcatctgtgcagggtgcagtttgattccttctggagtgctgatggctcagagcctggagcctgcttcggattctgtgtctcgcgctcgctctctgctcctcccccgctcatgctctgtctctctctgtctcagaaataaataaacattagaaaaaaagcaaagtatgatgcatgctcttccagctctcaaggagtccaccacctggagcgtggacgagagccattcctaggaaacccacacaagcctggtgtatgtatgtatagttgccgtatttcttcgccctctccacacacttgattcctgtgtgctaacagtcccctctctgtgtgcacttcccccccattagactgtggtttggtggcggttagggttaggttgctctgtgtcctctctttgtcaccctgcagactgtaaccagggacacaaccactcaggctgcctgacgctggaatctcgttcttatttcttggagaggtggagggtcaggagcgctaacccacgatacttgggtgataaacgctgaaaacaaacccaaaagcttttggtcttggaaacaatgttaggtgactattgcatttattttgtaaaaatctacccaacgcctccgcttgcctcttgggaggaacaaatgagaatacgaacgtgaaaataacttgaaaaactaaaaacgttacagaaataattaactcacttggaactgaaaaatgacactccatcgactcacttgtccatcctttccacaagcacccacgaggcaacgctgagcactctagatgcgtcgtactaagcgccaagagcagagggacgtgtccgtggggctctattctgggaggatcgtgttgttgggtaagcagacacctgctctttgcttccccacagggccggagagtgtgaggctggccgatggccccgggcgctgccagggccgagtggaggtgaagttccaaggggagtggagctctgtgtgccaagcaggctggagctttgcagccgccaaggtggtgtgccggcagctggggtgtggacgggccaccctgacccggagaggctgcaacaaagcgacccagggccaaggggccatctggcagagaaaggcgtcatgctcaggacaagaagtgagccttcaagattgcctttctgaagtttgggaacacaactgtacccacaatgaggacgtgtgggtcgaatgtgaaggtaatgcctatgagtccagaggcaggttcagtcatgaatttaggtcaatcggattcgctttcaccaaggctcacggagaacccgtcatccatcagatgacggtgacaaagttacttagctgtgctgcccatcaattctcttgtttttaaaatggagacaattggagagtctgggtggctcagtcggttaagcgtccagcttcggctcaggttatgatctcatggttcttgaactggagccccgtgttcaggtctgtgctgatggcttggggcctggaacggtcaaattctgcatctcccggtctctctgctcctcccctgttcatgctctgtctctgtctctctctcaaaaataaataaaacattaaaaaagttaaaatggagacaataatatgtgcccttgtatatggaggtgctttaagccatcttaaacactatattcatatgaactattattatattgaattctatttagtaggaacgggcacaccattccaggtattgttgagctcttcaattttttaaaaactagtggaaagtcatttagtaacgttagagtttttcacgtttttaaaaaatgtgtatttttgagagagagacagagcaaaagtgggggacggaagatccgaagcaggctctgccctgaacgcagtgagcccgacgcggggctcaaacccacgaaccttgagaccatgatttgaggcaaagtcagaggcttaactgactgagccacccaggtgcccctactgacgtcacattttaataaactagatggggaggtgggaaatcctggtgaaggaaagggaccagggccgcaaggtcaggtccagagaaggggatggagagtctcaggtgctgatgggatctgagtgccggctcagaggagcttacccatcatcctccctgcctcccgcccacccccctgttcctcctacacctgcagatccctttgccttgaagctggtaggaggacgcagccactgtgaggggaggctggaggtgctgcacaagggcgagtggggctctgtctgcgacgacggctggggacaagacgcagaccgggtggtgtgcaggcagctgggctgcgggcagcccctgtctccgcctgtcaaagtccggagaaggttcggccccggggtcggccgcatctggctggacgacgtcaagtgctcggggaaggagccgtccctggagcagtgcctgcacaggtcctggggctaccacaactgtaaccacagagaggatgtggctgtggtctgtgaaggtcagtcctgggtggacgggccagcgtggcgggaggggaggacagggggggctccctctgggggtgagaccccggctgccacgccgctcccccactttacgctttcttccagggacacgagtgaggccttccagccgtaaatagaaacaataacgatactgattattggtaaatcccaaaccgtctacctattgatgtgagagcaagggaggttttaaaaacggttggatcaacacccttagatcaaacacgaagagagtgagggtcagagaggataagcgactttccagaatcataaaaattgcttggaaatgatagagcagaacgaaagtgtcgggttctgactctgaactcaggggtcattgtcccagacgaccccagccctcgcaccctgactttaaactatgggagcctgtccctcctccttctacccgtgtgactcccttatttgaggtgaacccttgggaacggggctggggtagggggctctctggcaacgcccgggagacagagtcttctcagacagctggcagaccagccccgagctgcgggcgtctttgggggagggaaggaggaagccagaaaaatgcaggcacggcccctggggtccaaagctccgctctgtgtgcagggtagatatgcaggcctgctcgggtgaaacagtgccgtggggccgggtaagggccgacggggctgagcaaggcggtgacctctctttcctctctgggacccagtcttctctctgagccgtttagagcttgcatgaagccttccttctggacgtgctcccttctcactgtacatgcttcttctttctgcagaacagcagtctggcctacctgatgcttgacccacaggccccaagcgcccttacttctcctgggccctgatcggcccggcctgagccccccagccgcaggccgtacccacagccccgctctgcccccaggaagagtctgaactctgcctacgccttgctccagagccaagcgctcccgtggtcgcccggagttaccgggtgttgcaggccctccgccgggggagatgggccctcgtccccttgcccgcacagcggcccccctatcccggctggggcctctcctgcctcccctcgcccagcccggtgccctttgcagcacgggctgttgcccggccccatctctgtgcctgcaaagaacgtgaagggcgcaggatggttgcatggaacttggggggaaagcttagaaccttttaaactctttttctcacgcttcggacgaccacgacgattcatcgcacctcgccgccctcctgctcctcggtggcatcatgtcctttcagcagctgagcagcgtcctggaattctagaccctttcgtggcacctggaaaccatgttgcaccccctgaacgcctggaagaaaacggctctggtgaccagccagcgaggttgcggcctatcgagccgctgcgcgcacacacgcgtcctaacgggtcccaggagacgcgccggctgtgcggctgcgaccagaaggaggaagaggcccggggccgtgcctcacgccagtcagatggtgcctctcattttagcctctcacacactggcacctgcaagtctgaggcgacgcgcgaggctggtaaggagagaatcgacacttgattctaaaactcgagccttagactctgagctcctgtaatgtgcctgcatttacgccgcctaataaagtcgatctcacggtctaaacgtctcaa

SEQ ID NO: 2 - DNA, 320 bp, Felis catus
ggtctttttccagagtgcggctagtgggaggcgaccaccgctgtgaaggtcgtgtggagttgcagcaggatgacgagtgggtcaccgtgtgtgatgactactggaacatggactctgtggccgtgctgtgccgggagctgggctgtggggcggccaggaagaccatgagtggcaccgtgtatggaccagtgacaccaaaggaccaaaaagtcttcatccacctgttcagatgcaatgggatcgaagaaagcctgtctcagtgcgagagggaagatgcaatcggatgctcccatgttgaggatgcgggagccgtgtgcgag

SEQ ID NO: 3 - DNA, 320 bp, Felis catus
ggtctttttccagagtgcggctagtgggaggcgaccaccgctgtgaaggtcgtgtggagttgcagcaggatgacgagtgggtcaccgtgtgtgatgactactggaacatggactctgtggccgtgctgtgccgggagctgggctgtggggcggccaggaagaccatgagtggcaccgtgtatggaccagtgacaccaaaggaccaaaaagtcttcatccactcgttcagatgcaatgggatcgaagaaagcctgtctcagtgcgagagggaagatgcaatcggatgctcccatgatgaggatgtgggagtcgtgtgcgag

SEQ ID NO: 4 - DNA, 19 bp, Artificial Sequence
  Synthetic primer
tgaaggtcgtgtggagttg

SEQ ID NO: 5 - DNA, 22 bp, Artificial Sequence
  Synthetic primer
cacagagtccatgttccagtag

SEQ ID NO: 6 - DNA, 22 bp, Artificial Sequence
  Synthetic primer
caggatgacgagtgggtcaccg

SEQ ID NO: 7 - DNA, 20 bp, Artificial Sequence
  Synthetic primer
gatggctgattgctgtgaga

SEQ ID NO: 8 - DNA, 20 bp, Artificial Sequence
  Synthetic primer
cccaggaacctctgttcatt

SEQ ID NO: 9 - DNA, 20 bp, Artificial Sequence
  Synthetic primer
atcccggcttcggtcagctg

SEQ ID NO: 10 - DNA, 19 bp, Artificial Sequence
  Synthetic forward sense primer
gtgaaggtcgtgtggagtt

SEQ ID NO: 11 - DNA, 23 bp, Artificial Sequence
  Synthetic antisense probe
tcatcacacacggtgacccactc

SEQ ID NO: 12 - DNA, 22 bp, Artificial Sequence
  Synthetic reverse antisense primer
cacagagtccatgttccagtag

SEQ ID NO: 13 - DNA, 18 bp, Artificial Sequence
  Synthetic forwrad sense primer
tggacgacgtcaagtgct

SEQ ID NO: 14 - DNA, 20 bp, Artificial Sequence
  Synthetic antisense probe
cactgctccagggacggctc

SEQ ID NO: 15 - DNA, 22 bp, Artificial Sequence
  Synthetic reverse antisense primer
ccacatcctctctgtggttaca

SEQ ID NO: 16 - DNA, 20 bp, Artificial Sequence
  Synthetic forward sense primer
gagaactgtcagaagcagag

SEQ ID NO: 17 - DNA, 22 bp, Artificial Sequence
  Synthetic sense probe
caccaggaagccagacccactc

SEQ ID NO: 18 - DNA, 18 bp, Artificial Sequence
  Synthetic reverse antisense primer
gaagcggagtttggatgt

(19) United States
(12) Patent Application Publication
(10) Pub. No.: US 2022/0316007 A1
(43) Pub. Date: Oct. 6, 2022

(54) ASSOCIATION BETWEEN 4 COPIES OF EXON 3 OF fAIM AND PROGRESSIVE CHRONIC KIDNEY DISEASE IN CATS

(71) Applicant: Washington State University, Pullman, WA (US)

(72) Inventors: Liam BROUGHTON-NEISWANGER, PULLMAN, WA (US); Michael COURT, PULLMAN, WA (US); Nicolas VILLARINO, PULLMAN, WA (US); Neal BURKE, PULLMAN, WA (US)

(21) Appl. No.: 17/640,823

(22) PCT Filed: May 7, 2020
(86) PCT No.: PCT/US2020/031752

§ 371 Date: Mar. 6, 2022

Related U.S. Application Data
(63) Continuation of application No. PCT/US2019/051495, filed on Sep. 17, 2019.
(60) Provisional application No. 62/897,854, filed on Sep. 9, 2019.

(51) Int. Cl. C12Q 1/6883

(52) U.S. Cl. CPC C12Q 1/6883; C12Q 2600/156; C12Q 2600/118

(57) ABSTRACT

Individualized methods for the prevention and/or treatment of kidney disorders in cats are disclosed. The methods include identifying whether or not a cat is predisposed to developing kidney disorders by determining the total number of copies of Exon 3 in the feline apoptosis inhibitor of macrophages (fAIM) genes of the cat. Cats with at least 3 copies of Exon 3 are considered to be predisposed to kidney disorders to include tubulointerstitial fibrosis and/or Chronic Kidney Disease (CKD) and are provided with individualized kidney sparing therapies. Also disclosed is an in vitro amplification kit for determining the number of copies of Exon 3 in the feline apoptosis inhibitor of macrophages (fAIM) genes in a nucleic acid sample of interest.

### CROSS-REFERENCE TO RELATED APPLICATIONS

The instant application is a Continuation-in-part of International Patent Application No. PCT/US2019/051495, entitled “GENETIC TEST FOR IDENTIFYING CATS AT A HIGH RISK FOR DEVELOPING TUBULOINTERSTITIAL FIBROSIS.” filed Sep. 17, 2019 and claims the priority benefit of Provisional Application No. 62/897,854, entitled, “DEVELOPMENT OF A GENETIC TEST FOR IDENTIFYING CATS AT A HIGHER RISK FOR DEVELOPING TUBULOINTERSTITIAL FIBROSIS,” filed Sep. 17, 2019.

### FIELD OF THE INVENTION

The invention relates to Chronic Kidney Disease (CKD) in felines. In particular, the invention identifies the method to determine the predisposition of domestic and even non-domesticated cats for developing tubulointerstitial fibrosis, which can lead to progressive CKD.

### BACKGROUND OF THE INVENTION

Chronic kidney disease (CKD) is defined as a decline in renal filtration that persists at least three months. Plasma creatinine concentration is the most widely used marker of renal function. Once altered, kidney filtration can fully recover or remain altered (nonprogressive or stable). Alternatively, renal filtration can keep declining, resulting in progressive CKD. Multiple factors could influence the fate of altered kidney filtration. The inability to repair damaged kidneys with normal functional parenchyma is a well-established event that can lead to progressive CKD. Chronic kidney disease (CKD) can be classified in to different stages according to the severity of the disease and how the disease changes over time once diagnosed. Renal failure is considered the end-stage (worse state of CKD). Moreover, the disease can be classified as stable (when it does not get worse) or progressive when it gets worse over time (Polzin D. Chronic Kidney Disease, Textbook of Veterinary Internal Medicine, Ettinnger S. and Feldman E. Chapter 311 Vol 2, 1990-2020).

Chronic kidney disease (CKD) is the most common metabolic disease of domesticated cats and is a clinically important cause of mortality, especially in elderly domestic cats. It is estimated that as many as 35%-81% of cats older than 12 years-old are affected by CKD. Notably, however, all cats do not develop this disease. Even though there is an alarmingly high prevalence of kidney disease in the domestic cat population, the reason(s) underpinning why cats are remarkably prone to developing kidney disease is still an enigma. As a result of this important knowledge gap, there are currently no effective preventive measures or diagnostic tools which permit us to identify feline patients at risk for developing kidney disease before it is a problem, thus limiting the possibility of helping both cats and their owners.

One of the major causes of deleterious kidney changes in cats has been identified as long-term administration of Non-Steroidal Anti-inflammatory Drugs (NSAIDs). NSAIDs, such as meloxicam, are essential to treat pain and inflammation in many veterinary species. However, cats in particular are exquisitely sensitive to NSAIDs. The long-term administration of relatively high doses of NSAIDs can result in severe kidney changes associated with acute injury, including glomerular and tubular damage and interstitial lymphocytic infiltration.

A key aspect that characterizes NSAID-induced kidney disease in cats is the progression of kidney changes from early tubular damage to tubulointerstitial fibrosis. Early tubular damage results in accumulation of tubular luminal debris, predominantly composed of sloughed necrotic or apoptotic tubular epithelial cells. Recently, apoptosis inhibitor of macrophages (AIM), a key player in renal recovery from tubular insults, was described in cats. This protein is synthesized by tissue macrophages and circulates in plasma bound to immunoglobulins. Upon entering an injured renal tubule, AIM coats tubular debris and acts as the necessary ligand for phagocytosis of the debris, clearing the tubular lumen and paving the way for further renal repair and return to normal kidney function. However, in a subset of cats, failure to remove damaged cells and debris from the tubules favors the progression of kidney damage from a reversible process to irreversible tubulointerstitial fibrosis and lifelong negative renal changes, i.e. chronic kidney disease. In order to take steps to avoid the development of CKD, it is necessary to discover why some cats are less able to recover from early tubular damage and instead succumb to sustained damage to the glomerulus or tubule interstitium, progressive damage and loss of functional kidney mass. Regrettably, to date, these events remain clinically occult and undetected until there is a significant decrease in the functional renal mass that is detectable by traditional clinical markers.

Accordingly, there is a need to identify markers of the processes leading to the development of kidney disease so that vulnerable cats can be identified and treated at an early stage. The present invention addresses such a need by not only predicting fibrosis but also progressive CKD so as to connect both fibrosis and CKD.

### SUMMARY OF THE INVENTION

In humans, murine species and carnivores, AIM mRNA sequences are composed of 6 exons which code for a 37 kDa 3-domain AIM protein. Some cats also express a 3-domain AIM protein. However, others express either a 45 kDa 4-domain AIM protein or a combination of 3- and 4-domain AIM proteins. In vitro, both feline variants of AIM (fAIM) seem to have a similar pro-phagocytic effect. However, the 4-domain domain variant of fAIM is larger, less water soluble and more negatively charged than the 3-domain variant. Considering that all these features are not favorable for renal filtration, without being bound by theory, the present inventors hypothesized that the 4-domain variant of fAIM is not filtered through the glomerulus and is thus unable to enter injured renal tubules as efficiently as a 3-domain variant, hindering the crucial pro-phagocytic and tubular debris-clearing effect of AIM.

This disclosure provides evidence consistent with this hypothesis and identifies the changes in the cat genome that result in production of 4-domain AIM. A beneficial aspect of the technology is the discovery that a cat's susceptibility to tubulointerstitial fibrosis and/or CKD is based on the total number of copies of fAIM exon 3 present in the genome of the cat. A cat can have 2 copies of exon 3 (a “3 domain homozygote”), 3 copies of exon 3 (a “3 domain heterozygote”) or 4 copies of exon 3 (a “4 domain homozygote”). At the genomic level, cats of the “4 domain homozygote” genotype are at higher risk of having abnormal accumulation of collagen in renal parenchyma (tubulointerstitial fibrosis), and thus may be more susceptible to developing CKD. Cats of the “3-domain heterozygote” genotype may also be more susceptible.

The disclosure also provides methods for determining which AIM domain variant(s) a cat expresses at the genetic level (e.g. whether or not exon 3 is duplicated), thereby providing methods to determine whether or not a cat is prone to develop tubulointerstitial fibrosis and progressive Chronic Kidney Disease (CKD). By determining the AIM genotype of a cat, it is beneficially possible to implement treatment measures to prevent, delay the onset of and/or lessen symptoms associated with tubulointerstitial fibrosis (note that symptoms associated with fibrosis is what is known as renal failure and CKD), e.g., in cats that produce the 3-domain variant but that are homologous or the 4-domain variant of fAIM. In particular, it is possible to determine whether or not it is safe or advisable to use NSAIDs or any other nephrotoxic drug to treat pain in the cat or to implement any other intervention that could lead to CKD (e.g. any intervention that can result in hypotension and/or ischemia and reperfusion). Even more particular, the embodiments herein enable not only the likelihood of determination of developing fibrosis and progressive CKD by taking a genomic DNA sample, such as, but not limited to, any tissue sample that can include, a swab, a biopsy, a blood sample and/or a urine sample.

The present technology/embodiments herein significantly impact many areas of feline medicine. For example, it has an immense impact on how clinicians manage their patients, e.g., by enabling vets to implement kidney-sparing strategies in higher risk animals that could prevent or lessen the severe consequences associated with kidney disease, such as disability and death. The technology/embodiments herein also assist in the research and development of much needed treatments for cats with kidney disease. Further, the technology revolutionizes how owners take care of their pets and how breeders design breeding programs, e.g. for the selection of cats without this genetic predisposition. Also, the genetic tools described herein makes it possible to identify a population of cats at a low or high risk of tubulointerstitial fibrosis and thus benefits cats already suffering from kidney disease by assisting the selection of ideal kidney donors, helping both the donors and recipients for organ transplants.

Chronic kidney disease (CKD) affects 850 million people worldwide, including 15% of U.S. adults. However, there are no pharmacological therapies available to stop or reverse tubulointerstitial fibrosis (a key tissue abnormality of CKD) in humans. Innovative in-vivo models of tubulointerstitial fibrosis can reverse this situation and increase the clinical development success rate of drugs for treating CKD. Cats are an excellent model of naturally occurring tubulointerstitial fibrosis because some cats are prone to develop this condition and they share the same pathogenic mechanisms that leads to CKD in humans. Genotyping cats for AIM as described herein can be used as a key tool for generating an accurate model of tubulointerstitial fibrosis. This model can be a powerful, unique tool to study mechanisms of kidney repair and improve clinical development success rates for investigational drugs for humans, cats and other mammals.

The embodiments herein provide a method of identifying a cat at risk of developing tubulointerstitial fibrosis as well as identifying cats that will suffer from progressive CKD, and providing suitable preventive therapeutic measures and/or suitable treatment options to the cat, comprising: i) determining the number of copies of Exon 3 in the feline apoptosis inhibitor of macrophages (fAIM) genes in a nucleic acid sample from the cat; and ii) providing suitable preventive therapeutic measures and/or suitable treatment options to the cat when three or four copies of Exon 3 are present in the fAIM genes. In some aspects, the nucleic acid sample comprises genomic DNA. In some aspects, the suitable preventive therapeutic measures include: providing extra fluids to the cat; providing a special diet to the cat; and/or administering omega fatty acids to the cat. In further aspects, the suitable treatment options include administering non-nephrotoxic pain medication or treatments to the cat.

The disclosure also provides a method of treating pain and/or inflammation in a cat in need thereof, comprising i) determining the number of copies of Exon 3 in the feline apoptosis inhibitor of macrophages (fAIM) genes in a nucleic acid sample from the cat; and ii) administering at least one non-nephrotoxic therapy or an attenuated dose of a nephrotoxic agent to the cat when three or four copies of Exon 3 are present in the fAIM genes. In some aspects, the at least one non-nephrotoxic therapy includes administering to the cat one or more of: at least one non-nephrotoxic agent, laser therapy, stem cell therapy, acupuncture and a modified NSAIDs dosage regimen therapy. In further aspects, the at least one non-nephrotoxic agent is one or more omega fatty acids. In additional aspects, the nephrotoxic agent is a Non-Steroidal Anti-inflammatory Drug (NSAID). In some aspects, the methods further comprise a step of providing a kidney supportive therapy to the cat. In yet additional aspects, the kidney supportive therapy includes one or more of extra fluids, a special diet and administration of omega fatty acids.

The disclosure having supporting enabling data, also provides a method of treating or preventing kidney damage in a cat, comprising i) determining the number of copies of Exon 3 in the feline apoptosis inhibitor of macrophages (fAIM) genes in a nucleic acid sample from the cat (e.g. a DNA or RNA sample); and ii) providing a kidney supportive therapy to the cat when three or four copies of Exon 3 are present in the fAIM genes.

In some aspects, the kidney supportive therapy comprises one or more of: administering intravenous and/or subcutaneous fluids to the cat; providing a special diet to the cat; and administering omega fatty acids to the cat. To explain, cats can be genotyped at any age even younger than 5 years. In fact one of the benefits of the genetic test that is developed herein is that a cat 1 year old or younger can be tested so as to determine if that cat is a higher risk for developing progressive CKD later in the animals life, e.g., up to 10 years or longer. In some aspects, the cat is at least 5 years old. In additional aspects, the cat can be any breed, e.g., a domestic short hair or a domestic long hair cat.

The disclosure also provides a method of breeding cats, comprising i) determining the number of copies of fAIM Exon 3 that are present in the genome of a population of cats using the method of claim 1, and ii) breeding cats that have 2 copies of fAIM exon 3.

In addition, the disclosure provides a method of conducting a kidney transplant in a cat suffering from kidney disease, comprising i) determining the number of copies of fAIM Exon 3 that are present in the genome of a pool of potential donor cats using the method of claim 1; ii) removing a kidney from a cat that has 2 copies of fAIM exon 3; and iii) transplanting the kidney to the cat suffering from kidney disease.

Another aspect of the present disclosure provides for an in vitro amplification kit for determining the number of copies of Exon 3 in the feline apoptosis inhibitor of macrophages (fAIM) genes in a nucleic acid sample, wherein the kit includes: a DNA polymerase; dNTP's; one or more primers configured to bind to the nucleic acid sample and further configured to amplify a section of the nucleic acid sample that includes all or at least a portion of Exon 3 with or without flanking sequences; and at least one of: one or more buffers.

### BRIEF DESCRIPTION OF DRAWINGS

FIG. 1A-G shows histopathological examination of kidney tissue by HE staining. Histological damage scores (ranging between 0 and 5; means and SE) were based on percentage of tubules and glomeruli affected using 200 magnifications (0, no disease; 1, 1-25%; 2, 26-50%; 3, 51-75%; and 4, 76-100% of tissue affected). Sham treated animals did not show remarkable changes in cortical (A) and medullary tubules (C), while meloxican treatment induced severe tubula ectasia, epithelial necrosis, epithelial attenuation and proteinosis (B) and the cortical interstitium was multifocally infiltrated by lymphocytes, plasma cells and few neutrophils (D). Control animals did not show significant glomerular changes (E), compared with minimal unspecific changes observed in meloxicam group (F). Histopathological score is presented as mean±standard deviation (SD), n=4 meloxicam treated cats per group. *** p<0.001 vs. sham group (G).

FIG. 2 Shows the domain maps for feline variants of Apoptosis Inhibitor of Macrophages (fAIM). SRCR refers to scavenger receptor cysteine-rich domains.

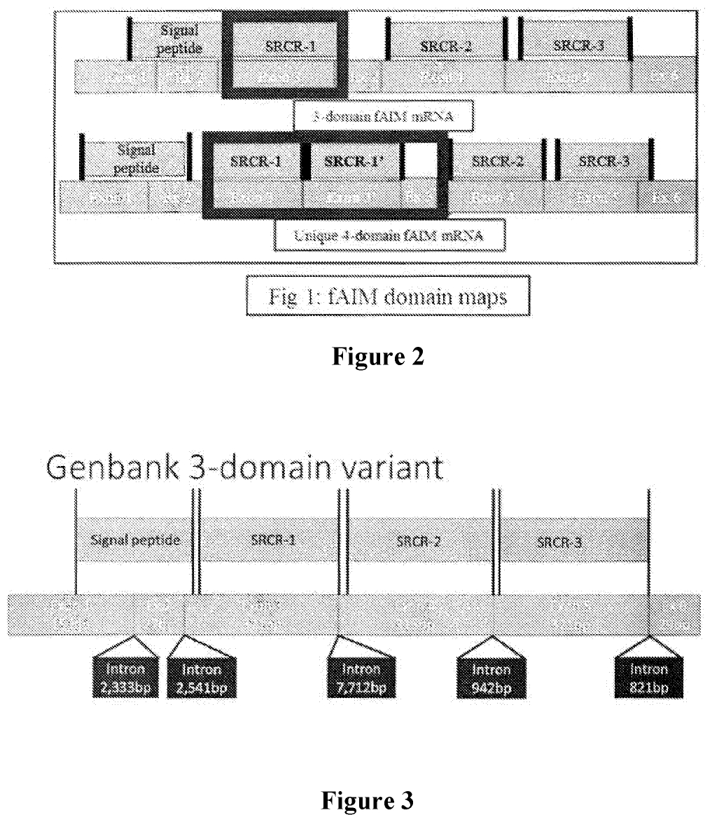

FIG. 3 shows a domain map of Genbank 3-domain variant.

FIG. 4 shows a domain map of NV-9 allele 1.

FIG. 5 shows a domain map of NV-9 allele 2.

FIG. 6 shows a domain map of NV-15 allele 1.

FIG. 7 shows a domain map of NV-15 allele 2.

FIG. 8 shows a domain map for 4-homozygote cat NV-10.

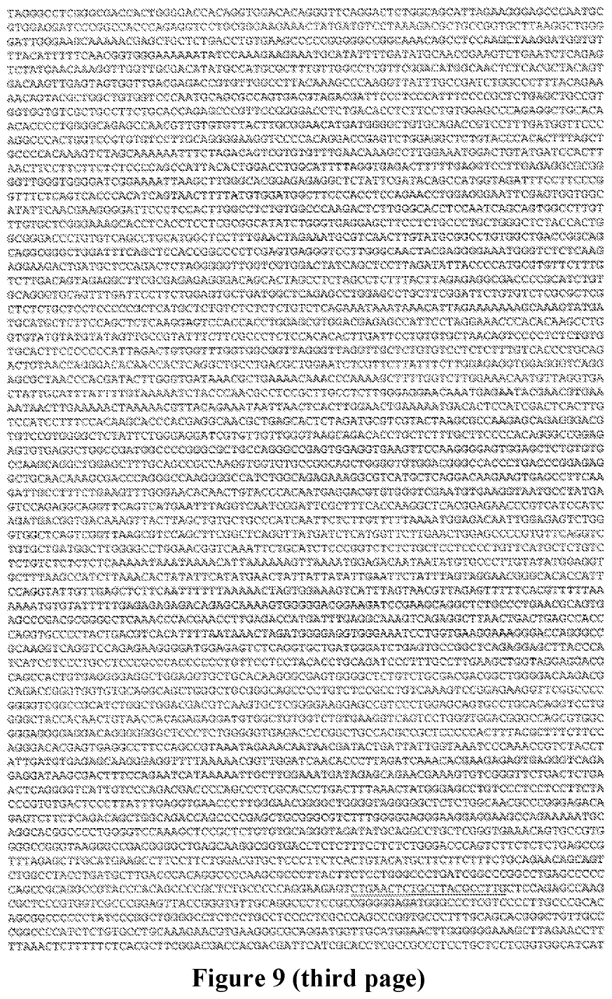

FIG. 9 shows a Nucleotide (nt) sequence of the fAIM gene (SEQ ID NO: 1; GenBank NC_018739.3:68680400-68696818 Felis catus isolate Cinnamon breed Abyssinian chromosome F1, Felis catus 9.0, whole genome shotgun sequence). The forward and reverse primer binding sites used in the present Examples are underlined.

FIG. 10 shows prediction of cats with progressive chronic kidney disease based on the number of copies of exon 3 of apoptosis inhibitor of macrophages (n=27) wherein bars showing increments in creatinine concentration >20% are indicative of progressive chronic kidney disease.

### DETAILED DESCRIPTION

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

While the invention has been described in terms of its example embodiments, those skilled in the art will recognize that the invention can be practiced with modification within the spirit and scope of the appended claims. Accordingly, the present invention should not be limited to the embodiments as described above but should further include all modifications and equivalents thereof with the spirit and scope of the description provided herein.

The present disclosure is directed towards identifying feline patients that are at risk for developing tubulointerstitial fibrosis, which predisposes them to CKD. In particular, the disclosure provides diagnostic methods and tools to identify cats that are likely to develop tubulointerstitial fibrosis, allowing the early detection and prevention and/or treatment of the disease, thereby lessening the chance of progression to CKD. Evidence is disclosed herein shows a prediction of both fibrosis and CKD using techniques, such as, for example, newer primers, newer reference genes, and a Polymerase chain reaction (PCR) technique, such as, but not limited to, digital droplet PCR.

Tubulointerstital fibrosis/CKD can be triggered by any factor or combination of factors that damage the renal tubules in cats, including but not limited to: administration of NSAIDs (especially in high doses), events that cause renal ischemia such as various surgical procedures, anesthesia related hypotension, administration of drugs (other than NSAIDs) which are known to be nephrotoxic in cats, exposure of cats to situations that alter renal blood flow such as dehydration, etc. The methods disclosed herein permit identification of susceptible cats before or after the occurrence of or exposure to one or more of these factors, and thus special prophylactic steps can be taken to mitigate or lessen the chance that the cat will develop tubulointerstitial fibrosis. In some aspects, the tubulointerstitial fibrosis is the result of exposure to NSAIDs, and methods are provided which permit the identification of cats that are especially susceptible to kidney damage by NSAIDs so that these agents are not used, or are used only sparingly, to treat pain and/or inflammation in the animal.

The methods disclosed herein involve determining the number of copies of exon 3 in the genome of a cat of interest. Without being bound by theory, it is believed that cats having 3 copies but more often 4 copies of exon 3 of fAIM (a “4 domain homozygote”) are not able to repair, or may be unable to efficiently repair, early tubular damage and thus are at higher risk for developing tubulointerstitial renal fibrosis, which may lead to CDK. For such cats, which appear to lack the ability to tolerate or recover from early tubular damage, the disclosure also provides steps that can be taken to avoid the development of and/or treat tubulointerstitial renal fibrosis, and thus slow or prevent progression to chronic kidney disease (CKD). For example, such cats may receive special diets, extra fluids, etc. as described in more detail below. In addition, in such cats, the use of nephrotoxic drugs such as NSAIDs is contraindicated and can be avoided, minimized or stopped; and the disease or condition that would otherwise be treated with the nephrotoxic drug (or that is already being or has previously been treated with the nephrotoxic drug) is instead treated with one or more suitable non-nephrotoxic agents or therapies. Alternatively, the dose of the nephrotoxic may be altered (usually decreased) and/or individually tailored to the cat e.g. in terms of the amount, frequency of administration, duration of administration, combination with other agents, etc. to avoid kidney damage, and other kidney-supportive treatment measures can be adopted. In addition, the present technology permits the pharmaceutical industry to include, in the labels of nephrotoxic drugs, the need and/or a recommendation to test cats for fAIM before a nephrotoxic drug is used. These and other aspects are discussed in detail below.

### Definitions

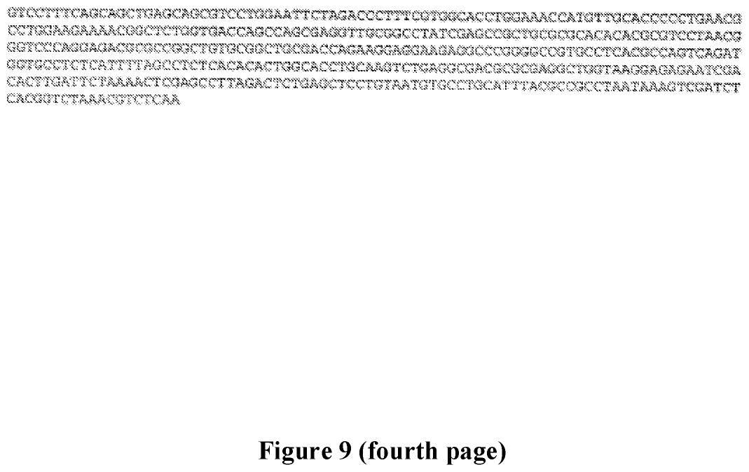

AIM (apoptosis inhibitor of macrophages, also called CD5-like antigen, CD5L) is encoded by the Cd51 gene. The most notable AIM function is in facilitating acute kidney injury repair via the enhancement of clearance of dead cell debris from the proximal tubules. An exemplary AIM gene sequence is listed as Genbank number NC_018739.3 and is shown in FIG. 9 and serves as the basis of nucleotide numbering used herein. Those of skill in the art will recognize that the exact sequence of a “fAIM gene” may vary somewhat from allele to allele, from cat to cat, from breed to breed, etc.

“Exon 3” of the AIM gene is located at nts 5080 to 5400 of the feline AIM Genbank sequence NC_018739.3. However, those of skill in the art will recognize that in some cats, or in some breeds of cats, the position of Exon 3 within the gene may vary somewhat, e.g. usually by only a few (e.g. about 1-5) nucleotides. In addition, the exact sequence of Exon 3 may vary somewhat from allele to allele, from cat to cat, from breed to breed, etc.

A contig (from contiguous) is a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequence and to overlapping physical segments (fragments) contained in clones depending on the context.

An exemplary Exon 3 sequence, in this case from allele 1 of cat subject NV-15, is shown below as SEQ ID NO: 2:

NV-15 allele 1 (exon 3)
(SEQ ID NO: 2)
GGTCTTTTTCCAGAGTGCGGCTAGTGGGAGGCGACCACCGCTGTGAAGG
TCGTGTGGAGTTGCAGCAGGATGACGAGTGGGTCACCGTGTGTGATGAC
TACTGGAACATGGACTCTGTGGCCGTGCTGTGCCGGGAGCTGGGCTGTG
GGGCGGCCAGGAAGACCATGAGTGGCACCGTGTATGGACCAGTGACACC
AAAGGACCAAAAAGTCTTCATCCACCTGTTCAGATGCAATGGGATCGAA
GAAAGCCTGTCTCAGTGCGAGAGGGAAGATGCAATCGGATGCTCCCATG
TTGAGGATGCGGGAGCCGTGTGCGAG

According to the invention, the number of copies of “Exon 3” of the fAIM gene are determined. Those of skill in the art will recognize that since the exact sequence of Exon 3 of the fAIM gene may vary somewhat from breed to breed, from cat to cat, or from allele to allele in one cat, etc., the sequences of the “Exon 3s” that are counted may differ from each other and/or from SEQ ID NO: 2 and still be considered “copies” of Exon 3. Such alternative Exon 3 sequences will generally exhibit e.g. at least about 75, 80, 85, 90 or 95% or more identity to SEQ ID NO: 2, and thus will be recognized by those of skill in the art as representing a copy of “Exon 3 of the fAIM gene”. For example, SEQ ID NO: 3 below shows the nucleotide sequence of Exon 3 from allele 2 of cat “NV-15”. SEQ ID NO: 3 has 96.4% nucleotide identity to SEQ ID NO: 2. While these two alleles can thus be differentiated by sequencing, the methods disclosed herein are based on determining the overall numbers of Exon 3 in the genome of a cat, regardless of the exact exon sequence. For example, cat NV-15 has 2 copies of fAIM exon 3 (one on each allele) indicating that this cat is homozygous for the smaller variant of fAIM and is likely at a lower risk for developing tubulointerstitial renal fibrosis.

Exon 3 from allele 2 of cat NV-15 (which may be
referred to herein as Exon 3')
(SEQ ID NO: 3)
GGTCTTTTTCCAGAGTGCGGCTAGTGGGAGGCGACCACCGCTGTGAAGG
TCGTGTGGAGTTGCAGCAGGATGACGAGTGGGTCACCGTGTGTGATGAC
TACTGGAACATGGACTCTGTGGCCGTGCTGTGCCGGGAGCTGGGCTGTG
GGGCGGCCAGGAAGACCATGAGTGGCACCGTGTATGGACCAGTGACACC
AAAGGACCAAAAAGTCTTCATCCACTCGTTCAGATGCAATGGGATCGAA
GAAAGCCTGTCTCAGTGCGAGAGGGAAGATGCAATCGGATGCTCCCATG
ATGAGGATGTGGGAGTCGTGTGCGAG

“Early tubular damage” that can lead to CKD is characterized by necrosis and/or apoptosis of tubular epithelial cells, and results in the accumulation of tubular luminal debris, composed predominantly of the sloughed necrotic or apoptotic tubular epithelial cells. Damage to tubular epithelial cells can be caused, for example, by NSAIDs or other nephrotoxic drugs such as aminoglycosides, vancomycin, imipenem, amphotericin B, cisplatin, carboplatin, methotrexate, doxorubicin, azathioprine, acyclovir, dapsone, apomorphine, cimetidine, deferoxamine, acetaminophen, diuretics, angiotensin-enzyme converting inhibitors, vitamin D, cyclosporine and tricyclic antidepressants. Failure to reverse the damage can lead to tubulointerstitial renal fibrosis and progressive CKD.

“Tubulointerstitial renal fibrosis” (tubulointerstitial fibrosis) is characterized as a progressive detrimental connective tissue (e.g. collagen) deposition on the kidney parenchyma, and is a harmful process leading to renal function deterioration, independently of the primary factor which causes an original kidney injury. It is noted that a cat can have tubulointerstitial fibrosis and still remain without clinical chronic disease. Tubulointerstital fibrosis can remain undetectable throughout the lifetime of the cat. However, in some cats the condition eventually becomes clinically evident and at that time the cat is considered to have CKD.

“Chronic kidney disease” (CKD) is present when there is long-standing, irreversible damage to the kidneys that impairs their ability to function and remove waste products from the blood. Affected kidneys often show a mixture of fibrosis and inflammation termed ‘chronic interstitial nephritis’. The cause may be idiopathic or well recognized, including: polycystic kidney disease (PKD), an inherited disease seen mainly in Persian and related cats where normal kidney tissue is gradually replaced by multiple fluid filled cysts; kidney tumors, for example lymphoma; bacterial infection of the kidneys (pyelonephritis); exposure to toxins and drugs such as NSAIDs; and glomerulonephritis, inflammation of the glomeruli. Symptoms of CKD include frequent urination, excessive drinking of water, bacterial infections of the bladder and kidney, weight loss and decreased appetite, vomiting, diarrhea, and bloody or cloudy urine, mouth ulcers, especially on the gums and tongue, bad breath with an ammonia-like odor, etc. CDK can also result in the death of the animal. Clinical markers include e.g. elevated serum creatinine (see data that utilizes such a marker) and urea concentrations, increased urine-specific gravity, increased serum symmetric dimethylarginine (SDMA), etc.

The phrase “kidney disorders” encompasses any type of kidney damage, e.g. early tubular damage, tubulointerstitial renal fibrosis and chronic kidney disease, and stages of these.

“Nonsteroidal anti-inflammatory drugs” (NSAIDs) are used to reduce pain, decrease fever, prevent blood clots and, in higher doses, decrease inflammation. NSAIDs are indicated as anti-inflammatory and analgesic agents and work by inhibiting the activity of cyclooxygenase enzymes (COX-1 and/or COX-2). In cells, these enzymes are involved in the synthesis of key biological mediators, namely prostaglandins which are involved in inflammation, and thromboxanes which are involved in blood clotting. Non-selective and COX-2 selective NSAIDs are available. Examples of NSAIDs that are sometimes used for the treatment of pain in cats include but are not limited to: robenacoxib, meloxicam, aspirin, carprofen, ketoprofen, tolfenamic acid, and the like.

MIQE is a set of guidelines that describe the minimum information necessary for evaluating qPCR experiments. Included is a checklist to accompany the initial submission of a manuscript to the publisher.

Detection of Cats with 4 Copies of Exon 3

As disclosed throughout the four corners of the present invention, methods of detecting cats with 4 copies of Exon 3 are encompassed by the present disclosure. While detecting 4 copies of Exon 3 is often an indicator, it is also to be appreciated, as stated above, that the methods herein provide that more than 2 copies, (e.g., 3 copies) of Exon 3 are also an indicator and can be detected as well for the prediction of maladies (e.g., fibrosis and CKD) discussed herein. The methods may include a step of identifying a cat, e.g., a cat that is to undergo surgery, a cat in need of treatment for pain and/or inflammation, etc. that would benefit from/is suitable for the practice of the method, e.g., a cat that is or has or is likely to experience one or more factors that could lead to tubulointerstital fibrosis or exacerbate the development of tubulointerstital fibrosis that can lead to CKD.

In addition, some cats such as, for example, Persian, Abyssinian, Siamese, Ragdoll, Burmese, Russian Blue and Maine Coon cats, are generally considered to be predisposed to CKD. These breeds often have 3 or 4 copies of Exon 3, and would benefit from the practice of the present methods. However, if the cat is not likely to experience such factors, obtaining the cat's genetic signature can still be beneficial for the animal's overall health care. Further, any cat or human associated with cats (e.g. cat owners, veterinarians, breeders, drug developers, etc.) can benefit from the knowledge provided by the methods described herein.

To practice the methods described herein, the genotype of a cat is determined with respect to the total number of copies of Exon 3 that are present in the fAIM gene, taking into account both alleles. If one copy of Exon 3 is present on both alleles, then the cat has 2 copies of exon 3, is a “3 domain homozygote” and produces only 3-domain AIM protein. Such cats are believed to be at a relatively low risk of developing tubulointerstital fibrosis. In some aspects, the risk level of such cats is used to establish a standard or reference value representing a “low” level of risk.

On the other hand, if two copies of Exon 3 are present on both alleles, then the cat has a total of 4 copies of Exon 3 and is a homozygous 4-domain variant. The cat then has a high risk of developing tubulointerstitial fibrosis and even CKD, most often progressive CKD, compared to a 3 domain homozygote, and thus may be more susceptible to kidney disease, most often tubulointerstitial fibrosis, CKD, and progressive CKD. In particular, treatment with nephrotoxic agents such as NSAIDs is more likely to cause tubulointerstitial fibrosis, which can lead to progressive CKD, compared to a 3-domain homozygous cat, and the use of such agents should be avoided (or at least kept to a minimum) and other kidney sparing treatment options should be used.

Alternatively, if two copies of Exon 3 are present on only one allele, and the other allele contains only one copy of Exon 3, then the cat has a total of 3 copies of Exon 3 and is a heterozygous 4-domain/3-domain variant (a “3 domain heterozygote”). The cat is thus capable of making at least some AIM without an Exon 3 duplication (3-domain AIM) but also likely produces some 4-domain AIM. Such cats may have a lower risk of developing tubulointerstitial fibrosis and progressive CKD than a homozygous 4-domain, but may still have a higher risk compared to a 2-domain homozygous cat. While AIM likely plays a role in the development of tubulointerstitial fibrosis and a predisposition to progressive CKD, development is multifactorial and can also depend on e.g., diet, life history of the cat, breed, anatomy, overall cat health, age, stress, etc. In addition, some heterozygous cats may be more at risk than other heterozygous cats, depending on the amount of 4-domain protein that they synthetize (due to e.g. differential expression). For 3 domain heterozygotes, the use of nephrotoxic agents and procedures should at least be carefully monitored and perhaps avoided, especially if other risk factors are present.

The genotype of the cat is generally determined by amplifying a section of the genome that comprises Exon 3 (or a portion of Exon 3, as described below) in a biological sample obtained from the cat. Suitable biological samples include but are not limited to: blood, serum, saliva, urine, cheek swabs, stool samples, liver, kidney and bone marrow biopsy samples, etc. Generally, a blood or cheek swab sample is employed. Nucleic acid (e.g. genomic DNA or fragments thereof, RNA) may be purified from the sample and prepared for amplification using known techniques, e.g. precipitation, centrifugation, resuspension in a suitable reaction buffer, etc.

Amplification of a nucleic acid molecule to increase the number of copies of a section of the nucleic acid molecule in a sample, e.g. by polymerase chain reaction (PCR), is known. The embodiments herein also capitalize on digital droplet PCR (ddPCR) amplification methodologies, as also known in the art. It is to be noted that such a ddPCR involves newer beneficial primers.

As an illustration, a sample containing DNA is contacted with at least two oligonucleotide primers (e.g., a plus or sense strand 5′—>3′ primer and a minus or antisense strand 3′—>5′ primer) under conditions that allow the primers to hybridize to sequences that flank a targeted region of interest within the DNA. The primers are extended under suitable reaction conditions, dissociated from the template, re-annealed, extended, and dissociated and so on repeatedly, to amplify the number of copies of the targeted region, thereby facilitating detection thereof. The amplification products (amplicons) are then quantified. If amplification products are detected, then the target sequence was present in the DNA. If no amplification products are detected, then the target sequence was not present. The quantity of amplicons that is produced can be used, in comparison to a suitable reference, to determine the number of copies of the targeted sequence that are present. Alternatively, the size (length) of an amplicon is indicative of the number of base pairs in the targeted region and can be used to identify, e.g. duplicated genes, insertions, deletions, etc. Amplicons can also be sequenced to determine the nucleotides that are present.

A key challenge of amplification is appropriate primer design. Those of ordinary skill in the art are generally familiar with primer design, including taking into account efficient annealing, optimal reaction temperature, etc. and with automated programs and services to design suitable primers, e.g. having a length of from about 15-30 nucleotide residues (bases), a G-C content between 40-60%, etc. However, a challenge in amplification experiments is often selecting which section(s) of a nucleic acid to amplify and then identifying suitable flanking sequences for primer binding. As seen in the Examples section below, for the present invention, this process was challenging and involved unexpected results. Optimization and validation of the qPCR primers was required, including: establishing temperature gradients to identify optimal annealing temperatures; establishing DNA concentration gradients to identify correct input DNA amounts and to establish linearity of the assay; the need to include DMSO in the reaction, etc.

For the present invention, one optimal region of DNA that is amplified comprises at least Exon 3, which includes nts 5080 to 5400 using the nucleotide numbering convention of the fAIM gene sequence shown in FIG. 9. However, it is also possible to detect Exon 3 duplication by amplifying regions that include more of the fAIM gene (e.g. regions which include sequences encoding part of one or more flanking exons such as Exons 2 or 4); or to amplify smaller sections of Exon 3. One or more of these regions can be amplified in a single reaction, or in separate reactions, in the practice of the method.

Alternatively, as this is a duplication of a segment of genomic DNA, a qPCR may be used to amplify the insertion site e.g. using a probe to bind over the insertion site of the duplicated exon. A signal would be obtained in a 3-domain homozygote and no signal would be obtained in a 4-domain homozygote, since the insertion would disrupt the sequence. Alternatively, a Droplet Digital™ PCR (ddPCR) assay may be used. ddPCR is a method for performing digital PCR that is based on water-oil emulsion droplet technology. Briefly, a sample is fractionated into 20,000 droplets, and PCR amplification of the template molecules occurs in each individual droplet.

Further, it is to be understood that the primers disclosed herein are exemplary only; other primers/probes can be designed to hybridize e.g. to alternate locations within the fAIM gene and still be used to successfully amplify Exon 3 or portions thereof. All such variations of the method are encompassed herein.

Generally, at least one reference gene or sequence in the cat genome that is not fAIM or part of fAIM is also amplified as a reference or internal control. Amplification of a second, known gene or sequence allows a practitioner to correlate the amount of amplicons produced from both loci, and to determine how many copies of Exon 3 are present. For example, if a single copy gene is used as a reference, the amount of amplicons produced from the Exon 3 region of a 3-domain homozygous cat will be substantially the same as that of the amount produced from the reference sequence (e.g. within an statistically acceptable margin of error). But if two copies of Exon 3 are present on both alleles as in a 4-domain homozygous cat, then the amount will be approximately twice (double) that of the single copy reference gene. If the duplication is present on only one allele (a 3-,4-domain heterozygous AIM) then the amount of amplicons will fall between that of a one copy gene and a two copy gene, e.g. the ratio of the amount of Exon 3 amplicons to reference amplicons will be about 1.5/1.0.

Examples of suitable reference sequences include but are not limited to: the albumin gene, or portions thereof sufficient to provide the requisite information; other genes that are known to consistently exist as single copy genes include RPP30 ribonuclease P/MRP subunit p30 and feline RPP30 (Gene ID 101083713), etc. When albumin (fALB) is used as the reference, the ratios of fAIM:fALB are as follows: 3-domain homozygous (1:1), 3-domain heterozygote (1.5:1), and 4-domain homozygous (2:1). However, those of skill in the art will recognize that genes known to have duplicate copies may also be used, so long as the relative numbers are adjusted accordingly, e.g. a genome with one copy of Exon 3/allele would yield half the number of amplicons as the reference, duplicated gene, and so on. Alternatively, or in addition, a synthetic reference sequence and/or or a gene sequence that is not from the cat genome, but which can be reliably correlated in terms of amount of amplicons produced, may be used. For example, synthetic nucleotide sequences of fAIM exon 3 may be used as a known copy concentration control.

Alternatively, or in addition, standardized reference values may be developed by correlating the quantity of amplicons produced from samples having a known number of copies of Exon 3 e.g. using a database of results from multiple experiments. Reference or control values may include a first reference value that is a numerical value, a range of values, and/or a cut-off value associated with the presence of e.g. one copy of Exon 3 per allele, and a second reference value that is associated with the presence of two copies of Exon 3 per allele, and a third reference value that is associated with the presence of two copies of Exon 3 on one allele and one copy of Exon 3 on the other allele, in a known quantity of DNA. In other words, cut-off or reference values can be established using a database of previously obtained data. Those of skill in the art are familiar with statistical analyses that can be used to extract meaningful, accurate reference values from experimental data.

Examples of in vitro amplification techniques that may be used in the practice of the invention include but are not limited to: quantitative real-time PCR; reverse transcriptase PCR (RT-PCR); real-time PCR (rt PCR); digital droplet PCR (ddPCR), real-time reverse transcriptase PCR (rt RT-PCR); nested PCR; strand displacement amplification (see U.S. Pat. No. 5,744,311); transcription-free isothermal amplification (see U.S. Pat. No. 6,033,881); repair chain reaction amplification (see PCT Publication No. WO 90/01069); ligase chain reaction amplification (see European patent publication No. EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134), amongst others. The complete contents of each of these references is herein incorporated by reference in entirety. Generally, the method that is used is digital droplet PCR or qPCR.

The products of amplification can be characterized and quantitated by such techniques as electrophoresis, restriction endonuclease cleavage patterns, oligonucleotide hybridization, ligation, and/or nucleic acid sequencing, dideoxy terminal and PACbio sequencing and combinations of these, as well as other techniques that are known to those of skill in the art. In some aspects, the amplification method that is used is PCR using Taq polymerase enzymes, as described in the Examples below.

### Treatment Options

In some aspects, if a cat is determined to have more than 2 copies of Exon 3, a skilled practitioner (e.g. a veterinarian) will implement kidney-sparing strategies to prevent, mitigate or halt progression of kidney disease. For example, a practitioner may recommend that NSAIDs or other drugs that have a deleterious impact on the kidney not be used to treat the cat, or that the amount that is administered be decreased. Procedures that reduce renal blood flow may be avoided or adjusted. For example, in some localities, cats are routinely neutered and spayed, exposing the cats to a combination of potential hypotension during surgery and treatment with NSAIDs after surgery, either or both of which may damage the kidneys. If this happens in cats with more than 2 copies of Exon 3 interstitial fibrosis may ensue and it would be beneficial to recognize the risk ahead of time and adjust the procedure and treatment accordingly.

As an example, a typical dose of the NSAID meloxicam for use in a cat is generally: for peri-operative use, a single injectable dose or 0.3 mg/kg body weight. It is noted that the FDA approved label for meloxicam has a black boxed warning message stating that the repeated administration should be avoided because it can result in kidney injury and death. Typically, for acute musculoskeletal disorders: on the first day of treatment, a single oral dose of 0.1 mg meloxicam/kg body weight (e.g. as an oral suspension) and thereafter once daily by oral administration (at 24 hour intervals) at a maintenance dose of 0.05 mg meloxicam/kg body weight for up to four days. For cats that are susceptible to developing tubulointerstitial fibrosis, a peri-operative dose may be lowered e.g. to 0.25, 0.2, 0.15, 0.1 or 0.05 mg/kg body weight, and optionally, an alternative pain medication can be utilized. For acute musculoskeletal disorders, the first day of treatment dose may be lowered e.g. to 0.05 mg mg/kg body weight and maintenance doses of e.g. 0.025 mg/kg body weight may be administered. In addition, or alternatively, the NSAID robenacoxib may be administered only once, or less than four days, e.g. for 1, 2 or 3 days.

Examples of anti-inflammatory and/or pain-relieving medications that can be used instead of (e.g. to replace) NSAIDs or other nephrotoxic agents, or to reduce (e.g. to partially replace) the amount of a nephrotoxic agent that is administered, include but are not limited to: opioids (e.g. codeine, fentanyl, hydromorphone, and morphine); corticosteroids (e.g. dexamethasone, prednisolone, methylprednisolone etc.); gabapentin; amitriptyline; the opiate partial agonist buprenorphine HCl; etc. Such agents may be especially useful to treat acute pain such that associated with an operation or acute injury but can also be used long-term to treat chronic pain, with caution, since side effects can also occur with these agents. There is robust evidence that administration of omega fatty acids, for example, but not strictly limited to, omega-3, can be successfully and efficaciously used to curb inflammation in cats and thus may be a good option to replace or partially replace nephrotoxic agents for example alone or in combination with low-dose NSAIDs therapy.

Examples of acute and chronic pain and/or inflammation relieving procedures that may be used to replace or partially replace conventional NSAID (or other nephrotoxic) therapy include but are not limited to: acupuncture, laser therapy, intermittent antifibrotic drugs, a modified NSAIDs dosage regimen therapy, such as low doses of NSAIDs (but the extent of the pain control would be limited and might require supplementation with another agent or therapy), etc. Such procedures and agents may be especially useful to treat chronic pain such that associated with arthritis.

Examples of measures that can be taken to mitigate the damage of nephrotoxic agents (e.g. that can be administered with nephrotoxic agents to reduce deleterious effects), or to treat damage that has already occurred due to the previous use of nephrotoxic agents (e.g. in cats that are identified as harboring the 4-domain AIM variant after administration of nephrotoxic agents) include but are not limited to: administration of intravenous and/or subcutaneous fluids (e.g. saline); special diets in which dry foods are avoided; special diets which are low in protein and/or phosphorous; administration of anti-inflammatory agents such as omega fatty acids; etc.

In other aspects, cats who benefit from the practice of the invention are not necessarily being considered for NSAID therapy, but knowledge of the cat's AIM gene signature is still beneficial. For example, if a cat is tested and identified as having a homozygous 4-domain variant AIM genotype, it may be possible to prevent, treat, lessen the chance of or delay the onset of one or more symptoms of kidney damage, e.g. early tubular damage, the accumulation of tubular luminal debris, tubulointerstitial fibrosis and full-blown CKD by adopting suitable measures. If a young cat is tested, e.g. a kitten less than 1 year old, it may be possible to treat the animal beneficially throughout its lifetime using the measures described elsewhere herein, e.g. extra fluids, special diets, etc. Such measures may improve the well-being of the cat and lengthen its lifespan. However, such measures may benefit cats of any age, e.g. cats that are 1-5, 5-10, 10-15, 15-20 or more years of age. Older (“senior”) cats (e.g. cats older than about 8) may especially benefit when such measures are adopted. In addition, felines cats of any gender or type may be assessed and treated as described herein, e.g., non-domestic cats (zoo felines and felines not in captivity: pumas, cheetahs, lions, tigers, etc.) and mixed breed domestic short- and long-haired cats or cats of an established breed (Siamese, Russian blue, Ragdoll, Maine coon, etc. and a plethora of others).

### Kits

Kits for determining the presence or absence of AIM Exon 3 duplication are also provided for a user, such as, for example, lab personnel (e.g., technicians, scientists, researchers, etc.) for conducting PCR and/or droplet ddPCR tests. Generally, such kits include inert pre-calibrated disposable and/or sterilizable droppers for liquid handling while performing nucleic acid amplification. The pre-calibration of such droppers enables precision and thus accurate amounts of droplets in the range of micro-liters up to milliliters.

Such kits may also include cartridges/containers preloaded with a control solution further including a target nucleic acid of interest. Such kits also often include cartridges/containers preloaded with desired disposed mixtures (e.g., one or more primers, buffers, polymerases, etc). Such disposed one or more primers (e.g., forward and reverse primers) are sequences of nucleic acid, complementary and capable of binding to a target nucleic acid sequence and that are suitable to amplify at least one appropriate section of the AIM gene e.g., a section that includes all or at least a portion of Exon 3 with or without flanking sequences. The kit may also include various cartridges/containers to provide disposed buffers (e.g., lysis buffers, wash buffers and elution buffers known in the art, ethanol, and aforementioned polymerases such as, for example, a high-fidelity long-range polymerase. The kit also often includes instructions for use.

It is also to be appreciated that the cartridges/containers can be labeled to avoid confusion. It is also to be noted that the cartridges/containers provided by the kit can be configured microtiter plates, micro-tubes, test tubes, or other containing means which does not react with fluids and solutions used in the embodiments herein. The kit also can include cartridges/containers preloaded with, for example Deoxynucleoside triphosphates (dNTP's) (e.g., dATP, dCTP, dGTP and dTT). In addition, the buffers preloaded in cartridges/containers can include but are not strictly limited to, Tris, EDTA, ammonium sulfate, potassium chloride, and stabilizers or other conventional buffers.

Moreover, while the aforementioned polymerase, i.e., a high-fidelity-long-range polymerase is disclosed, it is also to be noted that other DNA polymerases, such as, for example, a LongAmp® taq DNA polymerase, a thermophilic DNA polymerase, a recombinant DNA polymerase, and a modified DNA polymerase or other suitable polymerases can also be utilized herein without departing from the scope of the invention. The kit can also include the buffers (e.g., elution, wash, and lysis buffers) to be preloaded in cartridges/containers. The lysis buffers often include a detergent and a denaturant to break the cells apart and release nucleic acid molecules into the solution. The wash buffer can include a concentrated salt solution in a buffer such as TrisCl or its equivalent having a desired pH.

### Uses of the Technology

In addition to the uses described above (e.g. in breeding programs, in kidney transplant screening, etc.), the present technology can be used for or in a variety of additional endeavors. As an example, the tests described herein can be used in experimental design for drug development, e.g. by pharmaceutical companies. Using the test, it is possible to select or exclude 4-domain carriers for further testing e.g., in pre- and clinical development. Selected carriers are then used to test drug candidates for their ability to prevent, halt or reverse tubule interstitial fibrosis.

In addition, the tests can be used in the development of precision medicine and the individualization of treatment protocols. For example, the test can be used for assisting gene editing involving fAIM 4, e.g. to identify cats in need of gene editing therapy, to check the results of gene editing therapy, etc.

### EXAMPLES

### Example 1. A High Dose Regimen of Meloxicam Induces Acute Tubule Interstitial Nephritis

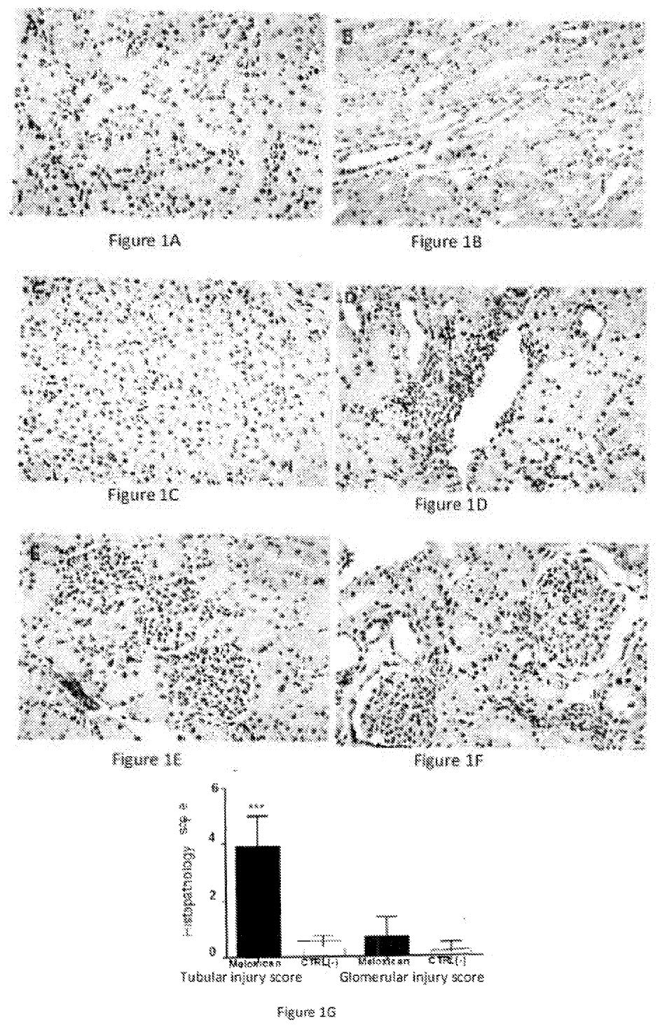

Histological assessment of replicated section of kidney revealed that meloxicam treatment induces severe tubulointerstitial nephritis. Kidney sections of negative controls were histologically unremarkable, showing complete Bowman's capsule, well-organized glomerulus, renal tubule with distributed brush border and regular nuclear arrangement (FIG. 1A, C, E). The histological changes in meloxican treated animals revealed severe intracytoplasmic vacuolation, tubular epithelial necrosis, and attenuation. There were also severe tubular ectasias and tubules containing slough necrotic epithelium and proteinaceous globules. Cortical tubules presented severe interstitial inflammation composed mostly of lymphocytes, plasma cells and a few neutrophils. Glomerular changes were minimal and were characterized by glomerular vacuolization, increased volume of the mesangial matrix, thickening of Bowman's capsule (glomerular parietal epithelium hyperplasia), and increased Bowman urinary space (FIG. 1B, D, F).

### Example 2. fAIM Genotyping: Identification and Analysis of Genome Features of Cats with the 4-Variant of fAIM

The genomic architecture which gives rise to the 4-variant of fAIM in cats was established using mRNA transcript data from a group of 12 cats. The odds of kidney disease in homozygous cats for 4-variant of fAIM were then determined using an aged-matched case control study design. mRNA transcripts: Briefly, mRNA was isolated from formalin-fixed paraffin embedded liver tissue samples in and converted to cDNA libraries. fAIM sequence specific primers, which amplify both 3- and 4-fAIM variants were used for amplification. The primer sequences for the fAIM pan-exon3 qPCR assay are as follows:

FP:
(SEQ ID NO: 4)
TGAAGGTCGTGTGGAGTTG;
RP:
(SEQ ID NO: 5)
CACAGAGTCCATGTTCCAGTAG;
and
Probe:
(SEQ ID NO: 6)
CAGGATGACGAGTGGGTCACCG.

The sequences that were targeted for amplification are shown in FIG. 9. PCR products were visualized with ethidium bromide on a 1% agarose gel. For 6 cats, PCR products for cDNA fAIM amplification were sequenced by the Sanger method to confirm that the visualized bands were indeed fAIM. The results of the fAIM cDNA predicted genotype for the group of 12 cats are presented in Table 1 below.

TABLE 1

| | cDNA predicted genotype |
|---|---|
| NV-9 | Heterozygous |
| NV-10 | 4-homozygous |
| NV-11 | Heterozygous |
| NV-12 | Heterozygous |
| NV-13 | Heterozygous |
| NV-14 | Heterozygous |
| NV-15 | 3-homozygous |
| NV-16 | 3-homozygous |
| NV-17 | Heterozygous |
| NV-18 | 3-homozygous |
| NV-19 | 3-homozygous |
| NV-20 | Heterozygous |

These results established the “true” genotype of each cat in the study, with respect to the 3- and 4-domain variants. Next, experiments were conducted to identify regions of the cat genome that, when PCR amplified, provided results that were in accord with the cDNA results. The amplification of such regions could serve as the basis of a rapid, inexpensive assay for identifying cats at risk of developing tubulointerstitial fibrosis, i.e. for identifying 4-homozygous cats. A large, —15 kb segment of genomic DNA was being amplified to enable a full length, single contig sequence. This allowed us to correctly identify the duplicated exon within the fAIM genomic architecture and to correlate the results with the predicted genotype. However, the PACbio sequencing was not straightforward and required special reaction conditions including the use of a high-fidelity-long-range polymerase and modification of reaction ingredients, including the inclusion of 5% DMSO.

### Example 3. Real-Time fAIM PCR Genotyping Assays

Pan-ex3/fAlbumin performed on DNA from fresh liver. The Taqman™ assay was redesigned with one probe specific for a known two-copy gene, feline albumin (GenBank NC_018726.3:c148423071-148407709 Felis catus isolate Cinnamon breed Abyssinian chromosome B1, Felis catus 9.0, whole genome shotgun sequence) and a second probe was designed specific for a conserved region shared between exon 3 and exon 3′ of the gene. The assay was based on that described by Helfer-Hungerbuehler et al. (Pseudogenes and the Quantification of Feline Genomic DNA Equivalents. Mol Biol Int. 2013; 2013:587680. Epub 2013/04/28. doi: 10.1155/2013/587680. PubMed PMID: 23738070; PubMed Central PMCID: PMCPMC3655645), with modifications.

The sequences of the primers and probe for fALB were:

FP:
(SEQ ID NO: 7)
GATGGCTGATTGCTGTGAGA;
RP:
(SEQ ID NO: 8)
CCCAGGAACCTCTGTTCATT;
and
Probe:
(SEQ ID NO: 9
ATCCCGGCTTCGGTCAGCTG.

Results from Taqman™ assay with Pan-ex3/fAlbumin performed on DNA from fresh liver from this assay were concordant with the cDNA predicted genotype for each cat, as shown in Table 2 below.

TABLE 2

| Sam- | Panex3 | fALB | | | | Exon 3 | Interpre- |
|---|---|---|---|---|---|---|---|
| ples | Cq mean | Cq mean | ΔΔCt | 2\hat{ }(−ΔΔCt) | Ratio | copies | tation |
| NV09 | 23.01 | 23.39 | —0.38 | 1.30 | 1.5 | 3 | Het |
| NV10 | 22.80 | 23.76 | —0.96 | 1.94 | 2 | 4 | 4-homo |
| NV11 | 22.98 | 23.47 | —0.49 | 1.41 | 1.5 | 3 | Het |
| NV12 | 23.16 | 23.49 | —0.33 | 1.26 | 1.5 | 3 | Het |
| NV13 | 23.25 | 23.67 | —0.43 | 1.35 | 1.5 | 3 | Het |
| NV14 | 23.01 | 23.48 | —0.47 | 1.39 | 1.5 | 3 | Het |
| NV15 | 23.13 | 23.16 | —0.03 | 1.02 | 1 | 2 | 3-homo |
| NV16 | 23.47 | 23.55 | —0.08 | 1.06 | 1 | 2 | 3-homo |
| NV17 | 22.98 | 23.65 | —0.67 | 1.59 | 1.5 | 3 | Het |
| NV18 | 23.66 | 23.88 | —0.22 | 1.16 | 1 | 2 | 3-homo |
| NV19 | 24.31 | 24.13 | 0.18 | 0.89 | 1 | 2 | 3-homo |
| NV20 | 22.57 | 23.24 | —0.67 | 1.59 | 1.5 | 3 | Het |

Pan-ex3/fAlbumin performed on DNA from formalin-fixed paraffin-embedded (FFPE) kidney: DNA yields retrieved from FFPE kidney tissues were adequate for the Taqman™ genotyping assay (average of 200 ng/μl) so a Taqman™ genotyping assay was attempted using primers shown above. The results showed a successful application to DNA samples isolated from FFPE. The results for DNA from formalin-fixed paraffin-embedded (FFPE) kidneys are presented in Table 3 below. While most of the results were in accord with the cDNA predicted genotype and fresh liver genotyping results, surprisingly one sample (NV-20) did not agree, yielding a 4-homologous result instead of a heterologous result.

TABLE 3

| Sam- | Panex3 | fALB | | | | ex3 | Interpre- |
|---|---|---|---|---|---|---|---|
| ples | Cq mean | Cq mean | ΔΔCt | 2\hat{ }(−ΔΔCt) | Ratio | copies | tation |
| NV09 | 30.67 | 31.31 | −0.64 | 1.56 | 1.5 | 3 | Het |
| NV10 | 28.55 | 29.54 | −1.00 | 1.99 | 2 | 4 | 4-homo |
| NV11 | 30.53 | 31.09 | −0.56 | 1.47 | 1.5 | 3 | Het |
| NV12 | 29.23 | 29.55 | −0.32 | 1.25 | 1.5 | 3 | Het |
| NV13 | 30.55 | 31.27 | −0.72 | 1.64 | 1.5 | 3 | Het |
| NV14 | 30.08 | 30.41 | −0.33 | 1.26 | 1.5 | 3 | Het |
| NV15 | 32.13 | 31.69 | 0.44 | 0.74 | 1 | 2 | 3-homo |
| NV16 | 31.89 | 31.76 | 0.13 | 0.91 | 1 | 2 | 3-homo |
| NV17 | 31.73 | 32.49 | −0.76 | 1.69 | 1.5 | 3 | Het |
| NV18 | 32.10 | 32.26 | −0.17 | 1.12 | 1 | 2 | 3-homo |
| NV19 | 31.32 | 31.53 | −0.21 | 1.15 | 1 | 2 | 3-homo |
| NV20 | 32.24 | 33.16 | −0.92 | 1.89 | 2 | 4 | 4-homo |

The Taqman™ fAIM genotyping assay was validated using the MIQE guidelines using both genomic DNA isolated from fresh liver and genomic DNA isolated from Formalin-Fixed Paraffin-Embedded (FFPE) blocks for cats NV 9-20.

### Example 4. Validation of the Pan-Ex3/fAlbumin fAIM Genotyping Assay

The fAIM genotyping assay was then applied to a retrospective case-control study set using archived FFPE feline kidney samples. Briefly, kidney samples from cats 5 to 10 years of age were selected based on the presence or absence of naturally occurring tubulointerstitial fibrosis (as opposed to tubulointerstitial fibrosis induced by NSAID use). Once a kidney sample had been chosen as a case or a control, the fAIM genotype was determined using the Pan-ex3/fAlbumin Taqman™ genotyping assay. The results for this study are shown below:

| | 4/4 | 3/3 | 3/4 |
|---|---|---|---|
| Tubulointerstitial fibrosis + | 12 | 2 | 3 |
| Tubuloinstertitial fibrosis − | 1 | 11 | 9 |

This data is consistent with the position that cats homozygous for the exon duplication (4/4) are 66× (CI: 5.2-833.6, p-value=0.0012) more likely to develop profound renal tubulointerstitial fibrosis than cats which lack the exon duplication (3/3 cats).

### Example 5. PacBio Sequencing

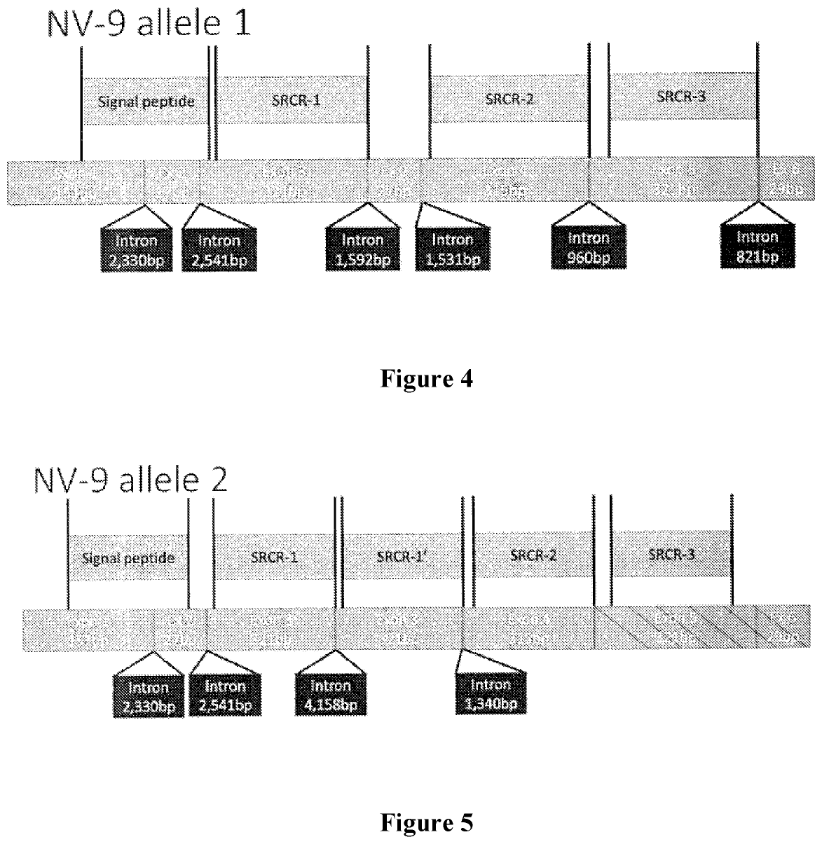

In order to further elucidate the genomic architecture of fAIM, a 15,628 kb fragment of genomic DNA (based on the Genbank sequence NC_018739.3 shown in FIG. 9) which encompasses exons 1-6 of fAIM (see FIG. 3) was amplified for the NV-9 (heterozygote) and the NV-15 (3-homozygote) cats using high fidelity, long range Taq polymerase enzymes. Briefly, this was done using a LONGAMP® Taq PCR kit from New England Biolabs. The protocol was slightly modified as described below. The products from the reaction were sequenced at the Washington State University molecular biology and genomics core using a PACbio sequencer.

| Component | 25 μl reaction | |
|---|---|---|
| 5x LongAmp taq rxn buffer | 5 | μl |
| 10 mM dNTPs | 0.75 | μl |
| 10 μM forward primer | 1 | μl |
| 10 μM reverse primer | 1 | μl |
| DMSO ([5%] final) | 1.25 μl of 100% DMSO | |
| LongAmp taq DNA polymerase | 1 | μl |
| Nuclease free-water | 10 | μl |

| | |
|---|---|
| Template DNA | 5 μl of 10 ng/μl DNA |

The two-step PCR cycle for this reaction was as follows:
Initial denaturation:
94° C. 30 seconds
30 cycles:
94° C. 30 seconds
65° C. 750 seconds (50 seconds/kb)
Final extension:
65° C. 10 minutes
Hold:
4° C.

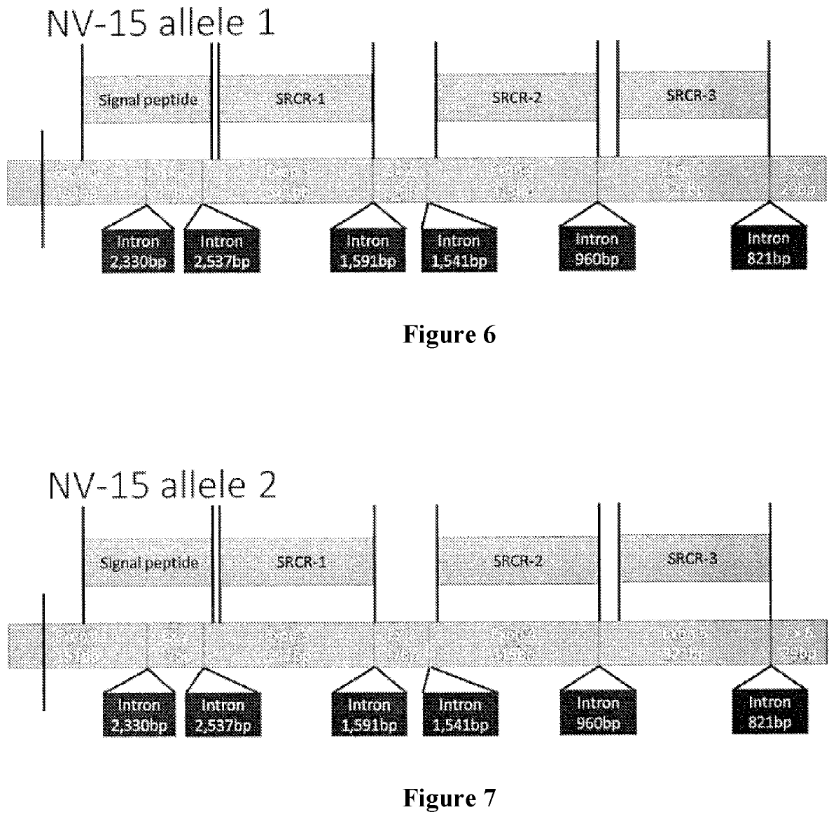

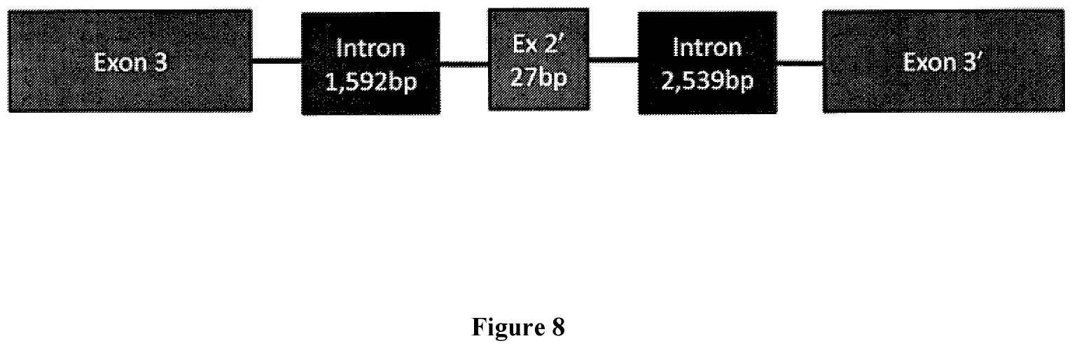

Data obtained in these studies showed that: In NV-9, the heterozygous cat, one allele is exon 3 alone (see FIG. 4) and the second allele contains exon 3 followed by a second copy of exon 3 (exon 3′) inserted within the intron between exon 3 and exon 4 (see FIG. 5). The cross hatching in the NV-9 allele 2 indicates that the sequence is likely present in the cat but was not amplified using this technique.

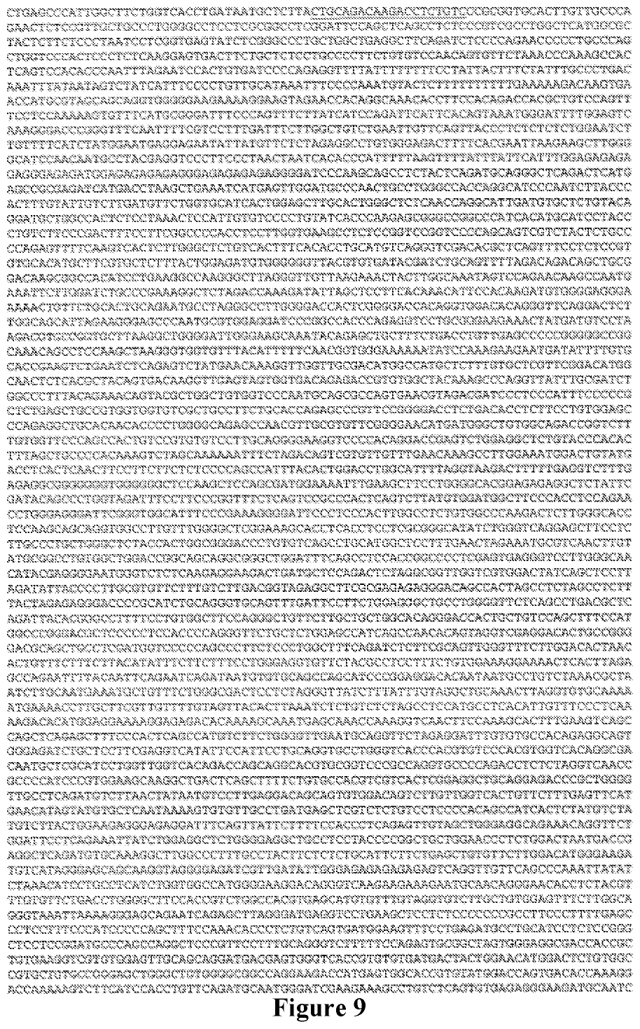

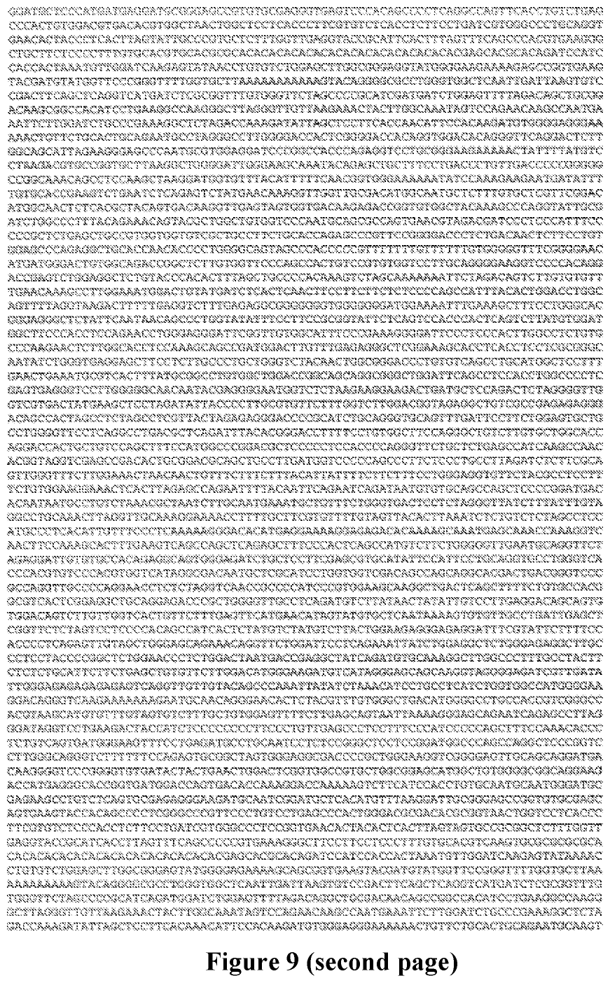

In addition, the results showed that, contrary to previous understandings, Exon 3′ can be present in the absence of exon 3. For example, in NV-15, one allele has exon 3 alone (FIG. 6) and the second allele has exon 3′ alone (FIG. 7). This finding explains the discordant results from the initial ex3/ex3′ genotyping assay described above.

### Droplet Digital PCR Technique for Detecting Exon 3 Duplication in Cats.

Droplet Digital PCR ddPCR is also disclosed as an example arrangement to detail the new cocktail of primers and probes for identifying cats fAIM exon 3 duplication.

### Materials and Methods

Genomic DNA (gDNA) was isolated from frozen liver from cats with known PacBio sequences and transcript profiles matching 3-domain fAIM variant homozygote (2 copies of exon 3) (cat NV15), 3-/4-domain variant heterozygote (3 copies of exon 3) (cat NV13) and 4-domain variant homozygote (4 copies of exon 3) (Cat NV10).

The gDNA was digested using HindIII restriction enzyme prior to ddPCR. The reaction mixture was composed of 2× Bio-Rad ddPCR mix, 100 ng of digested gDNA and 20× primer and TaqMan™ probe mixes for fAIM exon 3 as the region of interest (HEX). fAIM exon 5 and exon 3 of feline telomerase reverse transcriptase (TERT) were used as the reference gene (FAM) because are known to be present as a single copy. Primers Exon 3 and 5 of fAIM and TERT are listed below.

fAIM exon 3 probe was labeled with the fluorophore hexachlorofluorescein (HEX). The probes targeting the reference genes fAIM exon 5 and exon 3 TERT were labeled with the fluorophore fluorescein (FAM). The reaction mixtures were loaded into DG8 reaction cartridges (Bio-Rad, USA) with 70 μL droplet generation oil (Bio-Rad, USA), covered with a disposable gasket (Bio-Rad, USA) and placed in a droplet generator (Bio-Rad, USA). Following droplet generation, the droplets were transferred to a 96 well PCR plate (Bio-Rad, USA). PCR amplification was performed in a T100 Touch thermal deep-well thermocycler with the following parameters: 96° C. for 10 min followed by 45 cycles of 96° C. for 15 s and 60° C. for 2 min and finally 96° C. for 10 min and a 4° C. infinite hold all with a 2° C./s ramp rate. The PCR plate was cooled to room temperature prior to loading the plate in the QX200 digital droplet reader (Bio-Rad, USA).

The fluorescence data resulting was acquired and analyzed with QuantaSoft Analysis Pro Software version 1.05.596 (Bio-Rad, USA). The software calculates the copy number automatically. ddPCR software counts positive and negative droplets for each of the targets (fAIM exon 3 or reference gene) estimating the average number of copies number of fAIM exon3 per droplet.

TABLE 4
fAIM primer/probe sequences including in the test used for
estimation of fAIM exon 3 duplication at the genomic level.
fAIM EXON3
Amplicon Length: 77
Forward: GTGAAGGTCGTGTGGAGTT (Sense) (SEQ ID NO: 10)
Probe: TCATCACACACGGTGACCCACTC (AntiSense) (SEQ ID NO: 11)
Reverse: CACAGAGTCCATGTTCCAGTAG (AntiSense) (SEQ ID NO: 12)
fAIM EXONS
Amplicon Length: 96
Forward: TGGACGACGTCAAGTGCT (Sense) (SEQ ID NO: 13)
Probe: CACTGCTCCAGGGACGGCTC (AntiSense) (SEQ ID NO: 14)
Reverse: CCACATCCTCTCTGTGGTTACA (AntiSense) (SEQ ID NO: 15)
fTERT EXON3
Amplicon Length: 74
Forward: GAGAACTGTCAGAAGCAGAG (Sense) (SEQ ID NO: 16)
Probe: CACCAGGAAGCCAGACCCACTC (Sense) (SEQ ID NO: 17)
Reverse: GAAGCGGAGTTTGGATGT (AntiSense) (SEQ ID NO: 18)

### Results

The embodiments herein enable increased workings of the combination of primers and probes to allow the determination of the number of copies of fAIM exon3 in cats using ddPCR. In particular, the combination of primers and probes designed for this test identify cats carrying an fAIM exon3 duplication as shown in Table 4 below. In particular, Table 4 shows the number of copies of fAIM exon3 in cats using fAIM Exon 5 and TERT Exon 3 as reference gene.

TABLE 4

| | | | | Estimation of | | | |
|---|---|---|---|---|---|---|---|
| | | | | number of | | | |
| | | | | copies of | | | |
| | | | Estimation | fAIM exon3 | | | |
| | | | of number | using newer | | | |
| | | | of copies of | cocktail of | | | |
| | Genotype | | fAIM exon3 | primers and | | | |
| | classification based on | | using | probes | | Genotype | |
| | PacBio | transcript | albumin as | fAIM | TERT | based on | Kidney |
| Cat | sequence | profiles | reference gene | Exon 5 | Exon 3 | our tool | disease |
| NV15 | 3-domain | 3-domain | 2 | 2 | 2 | 2-domain | No |
| | fAIM | fAIM | | | | variant | |
| | variant | variant | | | | homozygote | |
| NV9 | 3-/4-domain | 3-/4-domain | 3 | 3 | 3 | 3/4-domain | No |
| | variant | variant | | | | variant | |
| | heterozygote | heterozygote | | | | homozygote | |
| NV10 | 4-domain | 4-domain | 4 | 4 | 4 | 4-domain | Yes |
| | variant | variant | | | | variant | |
| | homozygote | homozygote | | | | homozygote | |

Accordingly, an example cocktail of fAIM ex3 (HEX), fAIM EX5 (FAM), and TERT EX3 (FAM) primers and probes permitted identification of cats at a risk of kidney disease based on determining fAIM exon3 duplication in cats.

### Tool for Identifying Cat at a Higher Risk of Developing Progressive Chronic Kidney Disease

Kidneys are constantly exposed to different types of insults (e.g., nephrotoxic xenobiotics, ischemic conditions, etc.). Following an insult, renal repair mechanisms are initiated nearly immediately. Repair of renal lesions with normal parenchyma is known as adaptive repair. One key aspect that characterizes kidney disease is the progression of renal changes from damage to tubular epithelial cells to insidious interstitial fibrosis known as maladaptive repair, predisposing the patient to chronic kidney disease. Progression from the early stages of kidney disease depends on the balance of adaptive and maladaptive repair mechanisms. Clinically, some cats can improve or maintain stable kidney filtration function. Conversely, in some cats kidney filtration function worsens over time and they develop progressive chronic kidney disease.

Considering that maladaptive repair is a hallmark of chronic kidney disease, we hypothesize that cats carrying 4 copies of exon 3 in the apoptosis inhibitor of macrophages gene have higher odds of developing progressive CKD than cats carrying 2 copies of exon 3 in the same gene. We tested this hypothesis by determining the number of copies of exon 3 in the apoptosis inhibitor of macrophages gene in client-owned cats with abnormal plasma concentrations of creatinine admitted to the Veterinary Teaching Hospital at Washington State University using tools, such as, for example, ddPCR, and kits having necessary components to identify the number of copies of exon 3 of apoptosis inhibitor of macrophages.

### Materials and Methods

Selection of Medical Records of Cats with Progressive Chronic Kidney Disease

Medical records for cats admitted at the Veterinary Teaching Hospital at Washington State University between 2011 to 2017 were included in the study. The medical records were sorted based on the diagnosis of chronic kidney disease and the presence of collected DNA samples following the criteria described below.

A total of 142 medical records of cats diagnosed with kidney disease were retrieved from the medical records archive. Of those 142 medical records, 115 were excluded from the study because DNA samples were not collected from the corresponding patients or they did not meet the inclusion criteria listed below.

### Assessment of Kidney Function

Following the selection of medical records of patients diagnosed with kidney disease, plasma creatinine concentration data was retrieved from each patient's medical record. Cats with follow-up data on plasma creatinine concentration that did not exceed six months were excluded from the analysis to ensure an adequate time frame of follow-up data that would permit any subsequent changes in renal function to be identified. Twenty-seven medical records met the inclusion criteria.

Renal function was assessed by considering the International Renal Interest Society (IRIS) staging system criteria for kidney function staging, which is based on plasma creatinine concentration. Renal function was considered abnormal when the plasma creatinine concentration was >1.6 mg/dL.

### Classification of Patients According to Kidney Function

Based on the evolution of plasma creatinine concentration at diagnosis time, cats were classified into three categories with respect to their CKD status: progressive, stable, or non-progressive. Chronic kidney disease was considered as progressive when plasma creatinine concentration at the last recorded time increased at least 20% relative to the concentration at diagnosis. Stable CKD was considered when the plasma creatinine concentration at the last recorded time remained within 20% of the concentration at diagnosis. Chronic kidney disease was considered as non-progressive when the plasma creatinine at the last recorded time concentration decreased >20% relative to the concentration at diagnosis. The percentage of plasma creatinine concentration change was estimated by comparing the recorded creatinine concentration at the end of the follow-up period and diagnosis.

### Determination of the Number of Copies of Exon 3 in the Apoptosis Inhibitor of Macrophages Gene

DNA samples archived at WSU were used for determining the number of copies of exon 3 in the apoptosis inhibitor of macrophages gene. The presence of an exon 3 duplication in the apoptosis inhibitor of macrophages gene was determined using the tools described above and including, fAIM exon 3 probe labeled with the fluorophore hexachlorofluorescein (HEX) and probes targeting the reference genes fAIM exon 5 and exon 3 TERT labeled with the fluorophore fluorescein (FAM).

### Statistical Analysis

The strength of the association between progressive CKD and fAIM results was determined by calculating the odds ratio. An odds ratio greater than 1 indicated that progressive CKD and fAIM genotype results are associated. The association was tested statistically using Chi-Square. The significance level was set a<0.05. 95% confidence intervals are reported. Statistical analyses were done using R studio.

### Results

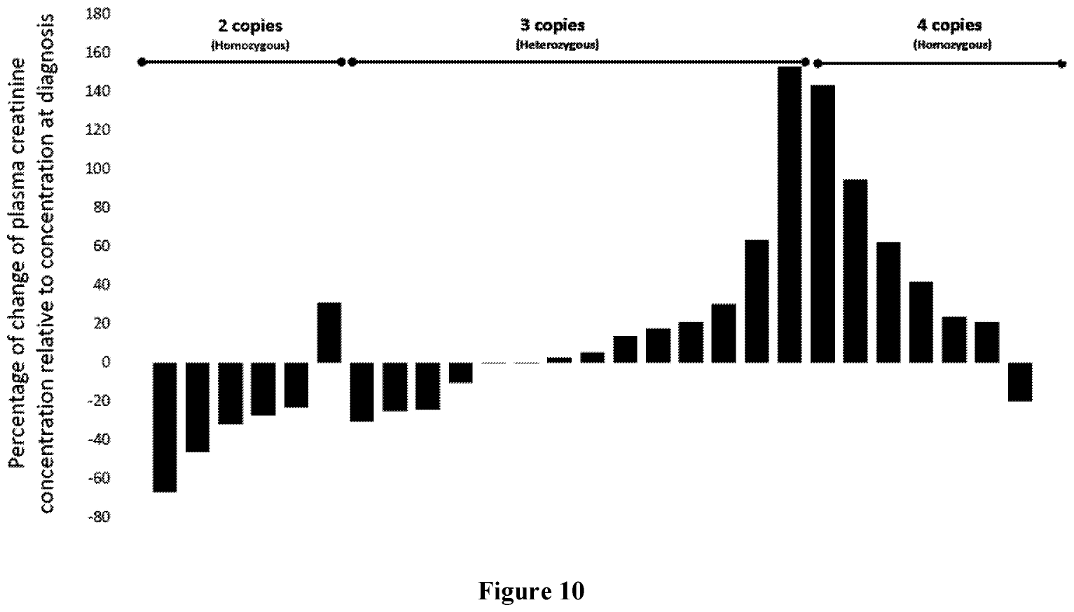

Five out of 6 (83%) cats with 2 copies of exon 3 in the apoptosis inhibitor of macrophages were able to reduce the plasma creatinine concentration (see Table 5 and Table 6, as shown below, and FIG. 10). FIG. 10 in particular shows prediction of cats with progressive chronic kidney disease based on the number of copies of exon 3 of apoptosis inhibitor or macrophages (n=27) wherein bars that indicate changes >20 are indicative of progressive chronic kidney disease. Table 5 below illustrates the association between copies of exon 3 of fAIM and progression of kidney function in cats diagnosed with chronic kidney disease.

TABLE 5

| | | | | | | | % of change |
|---|---|---|---|---|---|---|---|
| WSU | | | | | | | of creatinine |
| Medical | | | | | | | concentration |
| Record | Owners | | | | | | at last |
| Identification | Reported | Age at | | Color | FeSKI | CKD | recorded |
| Number | Breed | diagnosed | Gender | coat | Results | Status | time |
| 164120 | DSH | 16 | FS | black | − | Non- | −27 |
| | | | | & | | pCKD | |
| | | | | white | | | |
| 152900 | DSH | 11 | FS | brown | − | Non- | −32 |
| | | | | tabby | | pCKD | |
| 153036 | Ragdoll | 6 | MC | seal | − | pCKD | 31 |
| | | | | point | | | |
| 162198 | DSH | 10 | FS | black | − | Non- | −67 |
| | | | | | | pCKD | |
| 159539 | DSH | 12 | MC | orange | − | Non- | −27 |
| | | | | | | pCKD | |
| 141085 | Siamese | 10 | FS | tan and | − | Non- | −58 |
| | | | | black | | pCKD | |
| 129062 | Bengal | 5 | MC | brown | + | Stable | 0 |
| | | | | tabby | | | |
| 148558 | DSH | 7 | MC | black | + | pCKD | 64 |
| | | | | & | | | |
| | | | | white | | | |
| 149022 | DSH | 12 | MC | black | + | Stable | 5 |
| | | | | & | | | |
| | | | | white | | | |
| 152207 | DMH | 15 | MC | orange | + | pCKD | 153 |
| | | | | tabby | | | |
| 159607 | DSH | 11 | MC | brown | + | Non- | −30 |
| | | | | tabby | | pCKD | |
| 161954 | DSH | 13 | MC | grey | + | Stable | 21 |
| | | | | tabby | | | |
| 166427 | DSH | 13 | MC | orange | + | Stable | 17 |
| | | | | tabby | | | |
| 175689 | DSH | 5 | MC | black | + | Stable | 0 |
| 157350 | DLH | 15 | FS | grey | + | Stable | −10.3 |
| | | | | and | | | |
| | | | | white | | | |
| 159616 | DSH | 16 | mc | black | + | Stable | 2.7 |
| 129062 | feline | 8 | MC | tabby | + | Non- | −25 |
| | bengal | | | brown | | pCKD | |
| 104596 | DSH | 14 | MC | orange | + | Non- | −24. |
| | | | | white | | pCKD | |
| 148558 | DSH | 9 | MC | black | + | pCKD | 63 |
| | | | | and | | | |
| | | | | white | | | |
| 152078 | DLH | 7 | MC | Orange | + | Non- | 14 |
| | | | | | | pCKD | |
| 155764 | DSH | 15 | FS | Black | ++ | pCKD | 36 |
| | | | | and | | | |
| | | | | white | | | |
| 83256 | DSH | 7 | MC | Taby | ++ | pCKD | 21 |
| | | | | brown | | | |
| 140345 | Ragdoll | 13 | MC | blue | ++ | pCKD | 73 |
| | | | | point | | | |
| 140653 | Siamese | 15 | MC | cream | ++ | pCKD | 29 |
| | mix | | | | | | |
| 154927 | DSH | 15 | MC | black | ++ | pCKD | 143 |
| 173029 | DSH | 13 | FS | grey | ++ | Non- | −50 |
| | | | | tabby | | pCKD | |
| 174661 | DSH | 8 | MC | orange | ++ | pCKD | 95 |

DSH: domestic shorthair;
DMH: domestic medium hair;
MC: Male castrated;
FS: female spayed.
pCKD: progressive CKD it is considered when the plasma creatinine concentration increased >20% relative to the concentration at diagnosis (>1.6 mg/dL (corresponding to IRIS 1 stage)), stable: it so considered when the plasma creatinine concentration changed <20% relative to the concentration at diagnosis (>1.6 mg/dL (corresponding to IRIS 1 stage));
Non-pCKD Non-progressive CKD: it is considered when the creatinine concentration remains within 20% of the concentration at diagnosis. A negative value indicates that the latest creatinine concentration was lower than the concentration at diagnosis.
FeSKi (−): 2 copies of exon 3 of apoptosis inhibitor of macrophages (homozygous for a normal variant of the protein);
FeSKi (+): 3 copies of exon 3 of apoptosis inhibitor of macrophages (heterozygous);
FeSKi (++): 4 copies of exon 3 of apoptosis inhibitor of macrophages (homozygous for an abnormal variant of the protein).

The following are additional results to illustrate the association between 4 Copies of Exon 3 of fAIM and Feline Chronic Kidney Disease. Table 6 shows an association between copies of exon 3 of fAIM and progression of kidney function in cats diagnosed with chronic kidney disease.

TABLE 6
Table 6: Percentage of patients that were able reduced plasma
creatinine concentration at least 6 months after diagnosis.

| Genotype | CKD status | n= | % |
|---|---|---|---|
| FeSKi (−) | Non-progressive | 4 out 5 | 0.8 |
| FeSKi (+) | Non-progressive | 7 out 14 | 50 |
| FeSKi (++) | Non-progressive | 1 out 7 | 0.14 |

FeSKi (−): 2 copies of exon 3 of apoptosis inhibitor of macrophages (homozygous for normal variant of the protein); FeSKi (+): 3 copies of exon 3 of apoptosis inhibitor of macrophages (heterozygous); FeSKi (++): 4 copies of exon 3 of apoptosis inhibitor of macrophages (homozygous for abnormal variant of the protein).
Non-progressive: it is considered when the creatinine concentration decreased at least 20% relative to the concentration at diagnosis.
Progressive CKD (when plasma creatinine concentration at the last recorded time was at least 20% higher relative to the concentration at diagnosis).

One cat had reduced plasma creatinine concentrations at the last time of follow up testing of creatinine information data. Conversely, 6 out of 7 (85%) cats with 2 copies of exon 3 in the apoptosis inhibitor of macrophages had plasma creatinine concentration >20% higher at the end of the follow-up period relative to the time at diagnosis.

There was a statistical association (p<0.05) between the number of copies of exon 3 of apoptosis inhibitor of macrophages and the evolution of chronic kidney disease (non-progressive, stable and progressive). Table 7 shows the strength of the association between FeSKi results and in cats with naturally occurring chronic kidney disease.

TABLE 7

| 2 vs 4 copies of exon 3 | | | 3 vs 2 copies of exon 3 | | | 4 + 3 vs 2 copies of exon 3 | | |
|---|---|---|---|---|---|---|---|---|
| | | Non | | pCKD + | Non | | pCKD + | Non |
| | pCKD | pCKD | | Stable | pCKD | | stable | pCKD |
| FeSKi | n= | n= | FeSKi | n= | n= | FeSKi | n= | n= |
| (++) | 6 | 1 | (+) | 11 | 3 | (++) or | 17 | 4 |
| | | | | | | (+) | | |
| (-) | 1 | 5 | (-) | 1 | 5 | (-) | 1 | 5 |
| Odds | 30 | | Odds | 18 | | Odds | 21 | |
| ratio | (1.47-611) | | ratio | (1.5-222) | | ratio | (1.9-236) | |
| (95% | | | (95% | | | (95% | | |
| CI) | | | CI) | | | CI) | | |

| | | | | | |
|---|---|---|---|---|---|
| p= | 0.027 | p= | 0.022 | p= | 0.012 |

FeSKi (-): 2 copies of exon 3 of apoptosis inhibitor of macrophages (homozygous for the normal variant of the protein);
FeSKi (+): 3 copies of exon 3 of apoptosis inhibitor of macrophages (heterozygous);
FeSKi (++): 4 copies of exon 3 of apoptosis inhibitor of macrophages (homozygous for the abnormal variant of the protein).
pCKD: progressive CKD (when plasma creatinine concentration at the last recorded time was at least 20% higher relative to the concentration at diagnosis). The strength of the association between pCKD and FeSKi results was determined by calculating the odds ratio. Odds ratio greater than 1 indicated that pCKD and FeSKi are associated. Unadjusted odds ratio with 95% confidence interval (95 % CI) are reported. The association between pCKD and FeSKi results was tested statistically using Chi-Square. The significant level (p) was set a <0.05.

Accordingly, the results confirmed that the embodiments in the present application can be used to predict which cats have higher odds for developing progressive kidney disease.

The investigations described herein showed that cats expressing the 4-domain variant of fAIM are more likely to develop tubulointerstitial fibrosis, which favors the progression to abnormal changes in the kidneys' parenchyma, typically resulting in development of CKD. The results also showed that it is possible to quickly and reliably identify cats having the 4-domain fAIM variant using, for example, a Pan-ex3/fAlbumin fAIM genotyping assay.

It is to be understood that features described with regard to the various embodiments herein may be mixed and matched in any combination without departing from the spirit and scope of the invention. Although different selected embodiments have been illustrated and described in detail, it is to be appreciated that they are exemplary, and that a variety of substitutions and alterations are possible without departing from the spirit and scope of the present invention.

1. A method of identifying a feline at risk of developing tubulointerstitial fibrosis and Chronic Kidney Disease (CKD, comprising
determining the number of copies of Exon 3 in the feline apoptosis inhibitor of macrophages (fAIM) genes in a nucleic acid sample from the feline; and
identifying the feline as at risk of developing tubulointerstitial fibrosis and CKD when three or four copies of Exon 3 are present in the fAIM genes.

2. The method of claim 1, wherein the nucleic acid sample comprises genomic DNA.

3. The method of claim 21, wherein the suitable preventive therapeutic measures include: providing extra fluids to the feline; providing a special diet to the feline; and/or administering omega fatty acids to the feline.

4. The method of claim 21, wherein the suitable treatment options include administering non-nephrotoxic pain medication or treatments to the feline.

5. A method of treating pain and/or inflammation in a feline in need thereof, comprising
determining the number of copies of Exon 3 in the feline apoptosis inhibitor of macrophages (fAIM) genes in a nucleic acid sample from the feline; and
administering at least one non-nephrotoxic therapy or an attenuated dose of a nephrotoxic agent to the feline when three or four copies of Exon 3 are present in the fAIM genes.

6. The method of claim 5, wherein the at least one non-nephrotoxic therapy includes administering to the feline one or more of: at least one non-nephrotoxic agent, a laser therapy, an acupuncture therapy, a stem cell therapy, one or more antifibrotic drugs therapy, and a modified NSAIDs dosage regimen therapy.

7. The method of claim 6, wherein the at least one non-nephrotoxic agent is one or more omega fatty acids.

8. The method of claim 5, wherein the nephrotoxic agent is a Non-Steroidal Anti-inflammatory Drug (NSAID).

9. The method of claim 8, further comprising a step of providing a kidney supportive therapy to the feline.

10. The method of claim 9, wherein the kidney supportive therapy includes one or more of extra fluids, a special diet and administration of omega fatty acids.

11. A method of treating or preventing kidney damage in a feline, comprising
determining the number of copies of Exon 3 in the feline apoptosis inhibitor of macrophages (fAIM) genes in a nucleic acid sample from the feline; and
providing a kidney supportive therapy to the feline when three or four copies of Exon 3 are present in the fAIM genes.

12. The method of claim 11, wherein the kidney supportive therapy comprises one or more of: administering intravenous and/or subcutaneous fluids to the feline; providing a special diet to the feline; and administering omega-3 fatty acids to the feline.

13. The method of claim 11, wherein the feline is a feline selected from: a domestic and a non-domestic feline.

14. An in vitro amplification kit for determining the number of copies of Exon 3 in the feline apoptosis inhibitor of macrophages (fAIM) genes in a nucleic acid sample, wherein the kit comprises:
a DNA polymerase;
dNTP's;
one or more primers configured to bind to the nucleic acid sample and further configured to amplify a section of the nucleic acid sample that includes all or at least a portion of Exon 3 with or without flanking sequences; and
at least one of one or more buffers.

15. The in vitro amplification kit of claim 14, wherein the one or primers includes at least one of: a fAIM ex3 primer, a fAIM EX5 primer, and a TERT EX3 primer.

16. The in vitro amplification kit of claim 16 wherein the DNA polymerase is selected from: a high-fidelity-long-range polymerase, a thermophilic DNA polymerase, a recombinant DNA polymerase, and a genetically modified DNA polymerase.

17. The in vitro amplification kit of claim 14, wherein the dNTP's include at least one of: an ATP, a dCTP, a dGTP, and a dTTP.

18. The in vitro amplification kit of claim 14, wherein the buffers include at least one of: lysis buffers, wash buffers, and elution buffers.

19. The in vitro amplification kit of claim 16, wherein the in vitro amplification kit is configured for an vitro amplification technique selected from: a quantitative real-time PCR, a reverse transcriptase PCR (RT-PCR), a real-time PCR (rt PCR); a digital droplet PCR (ddPCR), a real-time reverse transcriptase PCR (rt RT-PCR), and a nested PCR.

20. The in vitro amplification kit of claim 16, wherein the DNA polymerase, the dNTP's, the one or more primers configured to bind to the nucleic acid sequence sample and further configured to amplify a section that includes all or at least a portion of Exon 3 with or without flanking sequences, and the at least one of one or more buffers; are provided in a respective labeled container.

21. The method of claim 1 further comprising a step of providing suitable preventative therapeutic measures and/or suitable treatment options to the feline.
